(19) United States
(12) Patent Application Publication
(10) Pub. No.: US 2015/0191651 A1
(43) Pub. Date: Jul. 9, 2015

(54) POLYMERIZABLE LIQUID CRYSTAL COMPOUND, LIQUID CRYSTAL COMPOSITION, POLYMER MATERIAL AND METHOD FOR MANUFACTURING THE SAME, AND FILM

(71) Applicant: FUJIFILM Corporation, Tokyo (JP)

(72) Inventors: Hiroshi MATSUYAMA, Kanagawa (JP); Shunya KATOH, Kanagawa (JP); Masaru YOSHIKAWA, Kanagawa (JP)

(73) Assignee: FUJIFILM Corporation, Tokyo (JP)

(21) Appl. No.: 14/665,349

(22) Filed: Mar. 23, 2015

Related U.S. Application Data
(63) Continuation of application No. PCT/JP2013/073288, filed on Aug. 30, 2013.

(30) Foreign Application Priority Data
Sep. 25, 2012 (JP) 2012-210377
Mar. 14, 2013 (JP) 2013-051319
Aug. 22, 2013 (JP) 2013-172610

(51) Int. Cl. C09K 19/04; C07C 233/55; G02B 5/30; C07D 251/54; C07D 213/72; G02F 1/1335; C07C 69/92; C07C 271/28

(52) U.S. Cl. CPC C09K 19/0403; C07C 69/92; C07C 233/55; C07C 271/28; C07D 251/54; C07D 213/72; G02F 1/133528; G02B 5/3016; C09K 2019/0407

(57) ABSTRACT

A polymerizable liquid crystal compound represented by the formula (1); wherein A¹ represents a C₂₋₁₈ methylene group, one CH₂ or two or more non-adjacent (CH₂)s in the methylene group may be substituted by —O—; Z¹ represents —CO—, —O—CO— or a single bond; Z² represents —CO— or —CO—CH═CH—; R¹ represents a hydrogen atom or methyl group; R² represents hydrogen, C₁₋₄ straight-chain alkyl group, C₁ or C₂ straight-chain alkoxy group, phenyl group, aryloxy group, vinyl group, acryloylamino group, methacryloylamino group, N-aryloxycarbamoyl group, N-alkyloxycarbamoyl group having a C₁₋₄ alkyl group, N-(2-methacryloyloxyethyl)carbamoyloxy group or N-(2-acryloyloxyethyl)carbamoyloxy group; and each of L¹, L², L³ and L⁴ independently represents C₁₋₄ alkyl group, C₁₋₄ alkoxy group, C₂₋₅ alkoxycarbonyl group, C₂₋₄ acyl group, halogen atom or hydrogen atom, at least one of L¹, L², L³ and L⁴ represents a substituent other than hydrogen atom.

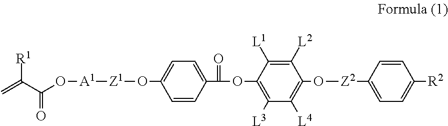

### CROSS-REFERENCE TO RELATED APPLICATIONS

This application is a Continuation of PCT International Application No. PCT/JP2013/073288 filed on Aug. 30, 2013, which claims priority under 35 U.S.C §119(a) to Japanese Patent Application No. 2012-210377 filed on Sep. 25, 2012, Japanese Patent Application No. 2013-051319 filed on Mar. 14, 2013, and Japanese Patent Application No. 2013-172610 filed on Aug. 22, 2013. Each of the above application(s) is hereby expressly incorporated by reference, in its entirety, into the present application.

### TECHNICAL FIELD

This invention relates to a polymerizable liquid crystal compound versatile for various applications, represented by various optical components including optically anisotropic film, heat barrier film and so forth; a liquid crystal composition using such polymerizable liquid crystal compound; a method of manufacturing a polymer material using such liquid crystal composition; a polymer material, and a film.

### BACKGROUND ART

Liquid crystal material has been used in various industrial fields including phase difference film, polarizing element, selective reflection film, color filter, antireflection film, viewing angle compensatory film, holography, alignment film and so forth. In particular, bifunctional liquid crystalline(meth)acrylate compound is highly versatile, and has been used for various applications.

The bifunctional liquid crystalline(meth)acrylate compound is, however, highly crystallizable, and single bifunctional liquid crystalline(meth)acrylate compound, or a composition of bifunctional liquid crystalline(meth)acrylate compounds, is unfortunately very likely to crystallize in a process of coating. It has therefore been desired to develop an additive which is effective to suppress crystal deposition of the polymerizable liquid crystal.

As a countermeasure, it has been known that mixing of a target polymerizable liquid crystal with other polymerizable liquid crystal compound successfully lowers the melting point. Patent Literature 1 also discloses that even crystallization may be suppressed by further mixing a polymerizable liquid crystal compound having a specific molecular structure. Patent Literature 1 describes that a liquid crystal material, having a bifunctional (meth)acrylate compound added thereto, in which the hydroquinone core having thereon a C₄ or longer substituent further has thereon a C₅ or longer substituent, is successfully suppressed from crystallizing even if super-cooled from the liquid crystal state down to room temperature, without degrading the characteristics including alignability and curability. Patent Literature 1, however, describes only bifunctional polymerizable liquid crystal compounds, and is unsatisfactory because the bifunctional polymerizable liquid crystal compound has a molecular structure having a poor-synthetic suitability which is needed to separately synthesize core moiety.

On the other hand, although not mentioned on suppression of the crystallization, Non-Patent Literature 1 describes a monofunctional polymerizable liquid crystal compound which is a benzoate ester of a substituted hydroquinone core. The monofunctional polymerizable liquid crystal compound described in Non-Patent Literature 1 was a compound configured by two different benzoate esters of methylhydroquinone, having a benzoate ester with a (meth.) acrylate group on one side, and having a benzoate ester with a C₆ alkoxy group on the other side. According to Non-Patent Literature 1, a cholesteric liquid crystal composition is manufactured by using a liquid crystal composition which contains 95% by mass of the above-described monofunctional polymerizable liquid crystal compound, 5% by mass of a chiral agent, and a polymerization initiator, so that there was no suggestion in Non-Patent Literature 1 about the use of the monofunctional polymerizable liquid crystal compound as an additive for suppressing crystallization.

Although not mentioned on suppression of the crystallization, also Patent Literature 2 describes a method for manufacturing a monofunctional polymerizable liquid crystal compound having a substituted hydroquinone core, as a random mixture with a bifunctional polymerizable liquid crystal compound. The monofunctional polymerizable liquid crystal compound contained in the random mixture described in Patent Literature 2 was a compound configured by two different benzoate esters of methylhydroquinone, having on one side a benzoate ester with a (meth)acrylate group, and having on the other side a benzoate ester with a C₄ alkoxy group as a side chain. Again in Patent Literature 2, neither disclosure nor suggestion was made on whether the compound described in the literature demonstrates a suppressive effect on crystallization.

### CITATION LIST

### Patent Literature

[Patent Literature 1] JP-A-2009-184974
[Patent Literature 2] Japanese Translation of PCT International Application Publication No. JP-T2-2002-536529

### Non-Patent Literature

[Non-Patent Literature 1] Molecular Crystals and Liquid Crystals (2010), 530 169-174

### SUMMARY OF THE INVENTION

### Technical Problem

While it has been generally known that the melting point of the target polymerizable liquid crystal lowers when mixed with other polymerizable liquid crystal compound, limited knowledge has been available on what kind of molecular structure of the compound, when added, is effective to suppress the crystallization, and this has been a matter of difficulty for prediction.

Considering such situation, the present inventors actually used the monofunctional polymerizable liquid crystal compound described in Non-Patent Literature 1 as an additive to study the suppressive effect on crystallization, and found only a small suppressive effect on crystallization. The present inventors also actually used the monofunctional polymerizable liquid crystal compound described in Patent Literature 2 to study the suppressive effect on crystallization and again found only a small suppressive effect on crystallization.

A problem to be solved by this invention relates to provide a polymerizable liquid crystal compound which is easily synthesized, and can demonstrate a high performance of suppressing the crystallization.

### Solution to Problem

After intensive studies aimed at solving the above-described problem, the present inventors found out that a polymerizable liquid crystal compound, in which a specifically-structured ester of a substituted hydroquinone core is configured as a laterally asymmetrical monofunctional compound, and the length of a substituent substituted on a phenyl group on the side containing no polymerizable group is made shorter than that of the compounds specifically disclosed in Patent Literature 2 and Non-Patent Literature 1, is weakened in crystallinity, and can therefore strongly suppress the crystallization of the other polymerizable liquid crystal compound.

The present invention aimed to solve the above-described problem is as described below.

[1] A polymerizable liquid crystal compound represented by the formula (1) below:

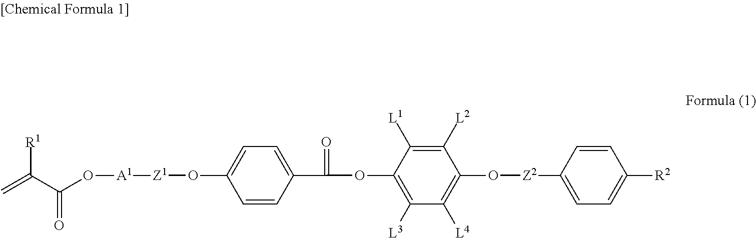

(wherein A¹ represents a C₂₋₁₈ methylene group, one CH₂ or two or more non-adjacent (CH₂)s in the methylene group may be substituted by —O—;
Z¹ represents —CO—, —O—CO— or a single bond;
Z² represents —CO— or —CO—C═C—;
R¹ represents a hydrogen atom or methyl group;
R² represents hydrogen, C₁₋₄ straight-chain alkyl group, C₁ or C₂ straight-chain alkoxy group, phenyl group, aryloxy group, vinyl group, acryloylamino group, methacryloylamino group, N-aryloxycarbamoyl group, N-alkyloxycarbamoyl group having a C₁₋₄ alkyl group, N-(2-methacryloyloxyethyl)carbamoyloxy group or N-(2-acryloyloxyethyl)carbamoyloxy group; and

each of L¹, L², L³ and L⁴ independently represents C₁₋₄ alkyl group, C₁₋₄ alkoxy group, C₂₋₅ alkoxycarbonyl group, C₂₋₄ acyl group, halogen atom or hydrogen atom, at least one of L¹, L², L³ and L⁴ represents a substituent other than hydrogen atom.)

[2] The polymerizable liquid crystal compound of [1], wherein in the formula (1), R² represents hydrogen, C₁₋₄ straight-chain alkyl group, C₁ or C₂ straight-chain alkoxy group or phenyl group.
[3] The polymerizable liquid crystal compound of [1] or [2], wherein each of L¹, L², if and L⁴ independently represents a C₁₋₄ alkyl group or hydrogen atom, and at least one of L¹, L², L³ and L⁴ represents a C₁₋₄ alkyl group.
[4] The polymerizable liquid crystal compound of any one of [1] to [3], wherein each of L¹, L², L³ and L⁴ independently represents a methyl group or hydrogen atom, and at least one of L¹, L², L³ and L⁴ represents a methyl group.
[5] The polymerizable liquid crystal compound of any one of [1] to [4], wherein each of L¹, L², L³ and L⁴ independently represents a methyl group or hydrogen atom, one of L¹, L², L³ and L⁴ being assigned to a methyl group and three of L¹, L², L³ and L⁴ being assigned to hydrogen atoms.
[6] The polymerizable liquid crystal compound of any one of [1] to [5], wherein Z¹ represents a single bond.
[7] The polymerizable liquid crystal compound of any one of [1] to [6], wherein A¹ represents a C₃₋₆ methylene group.
[8] The polymerizable liquid crystal compound of any one of [1] to [7], wherein A¹ represents a C₄ methylene group.
[9] The polymerizable liquid crystal compound of any one of [1] to [8], wherein Z² represents —CO—.
[10] The polymerizable liquid crystal compound of any one of [1] to [9], wherein R¹ represents a hydrogen atom.
[11] The polymerizable liquid crystal compound of any one of [1] to [10], wherein R² represents a C₁₋₄ straight-chain alkyl group, phenyl group, acryloylamino group or methacryloylamino group.
[12] The polymerizable liquid crystal compound of any one of [1] to [11], wherein R² represents a phenyl group, acryloylamino group or methacryloylamino group.
[13] A liquid crystal composition comprising

at least one polymerizable liquid crystal compound represented by the formula (1) below, and

at least one polymerizable liquid crystal compound represented by the formula (3) below:

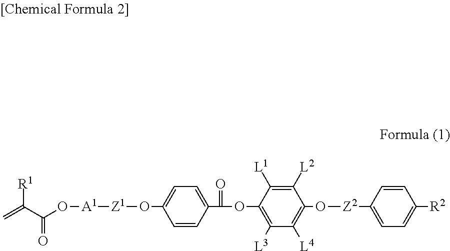

(in the formula (1), A¹ represents a C₂₋₁₈ methylene group, one CH₂ or two or more non-adjacent (CH₂)s in the methylene group may be substituted by —O—;
Z¹ represents —CO—, —O—CO— or a single bond;
Z² represents —CO— or —CO—C═C—;
R¹ represents a hydrogen atom or methyl group;
R² represents hydrogen, C₁₋₄ straight-chain alkyl group, C₁ or C₂ straight-chain alkoxy group, phenyl group, aryloxy group, vinyl group, acryloylamino group, methacryloylamino group, N-aryloxycarbamoyl group, N-alkyloxycarbamoyl group having a C₁₋₄ alkyl group, N-(2-methacryloyloxyethyl)carbamoyloxy group or N-(2-acryloyloxyethyl)carbamoyloxy group; and
each of L¹, L², L³ and L⁴ independently represents a C₁₋₄ alkyl group, C₁₋₄ alkoxy group, C₂₋₅ alkoxycarbonyl group, C₂₋₄ acyl group, halogen atom or hydrogen atom, at least one of L¹, L², L³ and L⁴ represents a substituent other than hydrogen atom.)

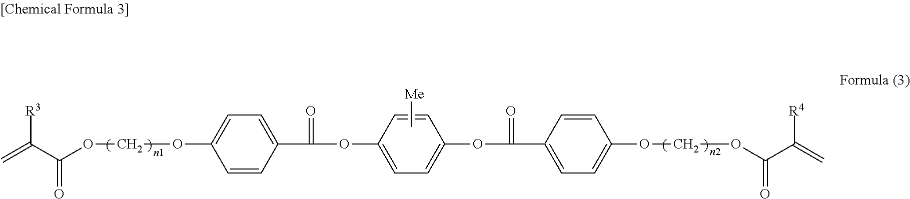

(in the formula (3),
each of n1 and n2 independently represents an integer of 3 to 6; and
each of R³ and R⁴ independently represents a hydrogen atom or methyl group.)
[14] The liquid crystal composition of [13], wherein in the formula (1), R² represents hydrogen, C₁₋₄ straight-chain alkyl group, C₁ or C₂ straight-chain alkoxy group or phenyl group.
[15] The liquid crystal composition of [13] or [14], wherein the polymerizable liquid crystal compound represented by the formula (1) is a polymerizable liquid crystal compound represented by the formula (2) below:

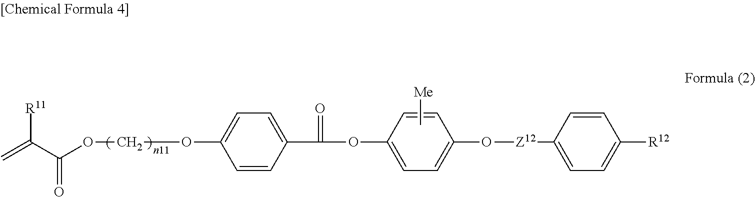

(in the formula (2),
n¹¹ represents an integer of 3 to 6;
R¹¹ represents a hydrogen atom or methyl group;
Z¹² represents —CO— or —CO—C═C—;
R¹² represents a hydrogen atom, C₁₋₄ straight-chain alkyl group, C₁ or C₂ straight-chain alkoxy group or phenyl group, aryloxy group, vinyl group, acryloylamino group, methacryloylamino group, N-aryloxycarbamoyl group, N-alkyloxycarbamoyl group having a C₁₋₄ alkyl group, N-(2-methacryloyloxyethyl)carbamoyloxy group or N-(2-acryloyloxyethyl) carbamoyloxy group.)
[16] The liquid crystal composition of [15], wherein n11 is 4.
[17] The liquid crystal composition of [15] or [16], wherein R¹ represents a hydrogen atom.
[18] The liquid crystal composition of any one of [15] to [17], wherein Z¹² represents —CO—.
[19] The liquid crystal composition of any one of [15] to [18], wherein R¹² represents a C₁₋₄ straight-chain alkyl group or phenyl group.
[20] The liquid crystal composition of any one of [15] to [19], wherein R² represents a phenyl group.
[21] The liquid crystal composition of any one of [13] to [20], which comprises the polymerizable liquid crystal compound represented by the formula (1) in a content of 3 to 50% by mass, relative to the polymerizable liquid crystal compound represented by the formula (3).
[22] The liquid crystal composition of any one of [13] to [21], which comprises the polymerizable liquid crystal compound represented by the formula (1) in a content of 5 to 40% by mass, relative to the polymerizable liquid crystal compound represented by the formula (3).
[23] The liquid crystal composition of any one of [13] to [22], containing at least one species of polymerization initiator.
[24] The liquid crystal composition of any one of [13] to [23], containing at least one species of chiral compound.
[25] A method for manufacturing a polymer material, the method comprising polymerizing the polymerizable liquid crystal compound described in any one of [1] to [12], or the liquid crystal composition described in any one of [13] to [24].
[26] The method for manufacturing a polymer material of [25], wherein the polymerization is allowed to proceed under ultraviolet irradiation.
[27] A polymer material configured by polymerizing the polymerizable liquid crystal compounds described in any one of [1] to [12], or the liquid crystal composition described in any one of [13] to [24].
[28] A film comprising at least one species of the polymer material described in [27].
[29] A film comprising an optically anisotropic layer, wherein the optically anisotropic layer is obtainable by fixing alignment of polymerizable liquid crystal compounds described in any one of [1] to [12], or polymerizable liquid crystal compounds in a liquid crystal composition described in any one of [13] to [24].
[30] The film of [29], wherein the optically anisotropic layer is obtainable by fixing cholesteric alignment of the liquid crystal compounds.
[31] The film of [30], having selective reflectivity.
[32] The film of [30] or [31], having selective reflectivity in an infrared wavelength region.
[33] The film of [29], wherein the optically anisotropic layer is obtainable by fixing homogeneous alignment of the liquid crystal compounds.
[34] The film of [29], wherein the optically anisotropic layer is obtainable by fixing homeotropic alignment of the liquid crystal compounds.
[35] A polarizing plate comprising the film described in [33] or [34], and a polarizing film.
[36] A liquid crystal display device comprising the polarizing plate described in [35].

### Advantageous Effects of Invention

According to this invention, it is now possible to provide a polymerizable liquid crystal compound which is easily synthesized, and can demonstrate a high performance of suppressing the crystallization.

### DESCRIPTION OF EMBODIMENTS

This invention will be detailed below. Explanation of constituent features will occasionally be made on representative embodiments or specific examples of this invention, to which this invention by no means limited. In this specification, all numerical ranges expressed using “to” with preceding and succeeding numerals are defined to contain these numerals as the lower and upper limit values.

In this specification, (meth)acrylate means a group consisting of both of acrylate and methacrylate.

### [Polymerizable Liquid Crystal Compound]

The polymerizable liquid crystal compound of this invention is characteristically represented by the formula (1) below:

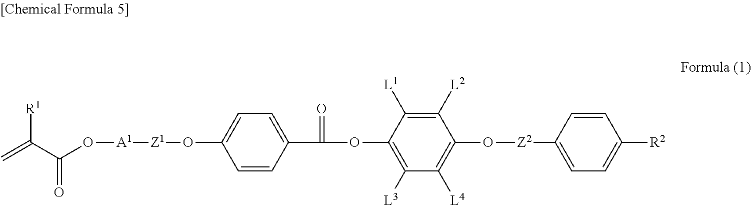

(in the formula (1), A¹ represents a C₂₋₁₈ methylene group, one CH₂ or two or more non-adjacent (CH₂)s in the methylene group may be substituted by —O—;
Z¹ represents —CO—, —O—CO— or a single bond;
Z² represents —CO— or —CO—C═C—;
R¹ represents a hydrogen atom or methyl group;
R² represents hydrogen, C₁₋₄ straight-chain alkyl group, C₁ or C₂ straight-chain alkoxy group, phenyl group, aryloxy group, vinyl group, acryloylamino group, methacryloylamino group, N-aryloxycarbamoyl group, N-alkyloxycarbamoyl group having C₁₋₄ alkyl group, N-(2-methacryloyloxyethyl) carbamoyloxy group or N-(2-acryloyloxyethyl) carbamoyloxy group;
each of L¹, L², L³ and L⁴ independently represents a C₁₋₄ alkyl group, C₁₋₄ alkoxy group, C₂₋₅ alkoxycarbonyl group, C₂₋₄ acyl group, halogen atom or hydrogen atom, and at least one of L¹, L², L³ and L⁴ represents a substituent other than hydrogen atom.)

The polymerizable liquid crystal compound of this invention having such structure is easily synthesized, and a good performance of suppressing the crystallization.

A¹ represents a C₂₋₁₈ methylene group, and one CH₂ or two or more non-adjacent (CH₂)s in the methylene group may be substituted by —O—.

A¹ preferably represents a C₂₋₇ methylene group, A¹ more preferably represents C₃₋₆ methylene group, and A¹ particularly represents a C₄ methylene group. While one CH₂ or two or more non-adjacent (CH₂)s in the methylene group may be substituted by —O—, the number of (CH₂)s substituted by —O— in the methylene group is preferably 0 to 2, more preferably 0 or 1, and particularly 0.

Z¹ represents —CO—, —O—CO— or a single bond, and preferably represents a single bond.

Z² represents —CO— or —CO—C═C—, and preferably represents —CO—.

R¹ represents a hydrogen atom or methyl group, and preferably represents a hydrogen atom.

R² represents hydrogen, C₁₋₄ straight-chain alkyl group, C₁ or C₂ straight-chain alkoxy group, phenyl group, aryloxy group, vinyl group, acryloylamino group, methacryloylamino group, N-aryloxycarbamoyl group, N-alkyloxycarbamoyl group having a C₁₋₄ alkyl group, N-(2-methacryloyloxyethyl)carbamoyloxy group or N-(2-acryloyloxyethyl)carbamoyloxy group, preferably represents C₁₋₄ straight-chain alkyl group, phenyl group, acryloylamino group or methacryloylamino group, and more preferably represents phenyl group, acryloylamino group or methacryloylamino group.

The C₁₋₄ straight-chain alkyl group preferably has 1 to 3 carbon atoms, and more preferably has 2 carbon atoms.

The C₁ or C₂ straight-chain alkoxy group preferably has one carbon atom.

The N-alkyloxycarbamoyl group having a C₁₋₄ alkyl group preferably has 2 to 4 carbon atoms.

In the polymerizable liquid crystal compound of this invention, each of L¹, L², L³ and L⁴ independently represents a C₁₋₄ alkyl group or hydrogen atom, and at least one of L¹, L², L³ and L⁴ is preferably a C₁₋₄ alkyl group.

Each of L¹, L², L³ and L⁴ independently represents a straight-chain C₁ or C₂ alkyl group or hydrogen atom, and at least one of L¹, L², L³ and L⁴ more preferably represents a C₁ or C₂ alkyl group.

Each of L¹, L², L³ and L⁴ independently represents a methyl group or hydrogen atom, and at least one of L¹, L², L³ and L⁴ preferably represents a methyl group.

In the polymerizable liquid crystal compound of this invention, each of L¹, L², L³ and L⁴ independently represents a methyl group or hydrogen atom, wherein it is more preferable that one of L¹, L², L³ and L⁴ is assigned to a methyl group, and three of L¹, L², L³ and L⁴ are assigned to hydrogen atoms.

The polymerizable liquid crystal compound of this invention is preferably a polymerizable liquid crystal compound represented by the formula (2) below:

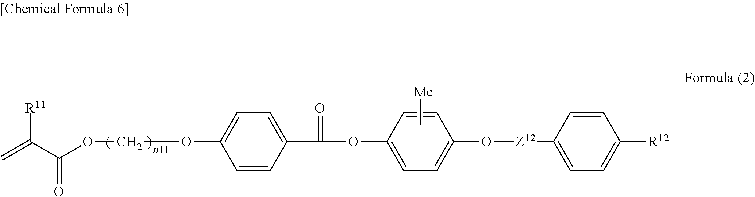

(in the formula (2),
n11 represents an integer of 3 to 6;
R¹¹ represents a hydrogen atom or methyl group;
Z¹² represents —CO— or —CO—C═C—;
R¹² represents a hydrogen atom, C₁₋₄ straight-chain alkyl group, C₁ or C₂ straight-chain alkoxy group, phenyl group, aryloxy group, vinyl group, acryloylamino group, methacryloylamino group, N-aryloxycarbamoyl group, N-alkyloxycarbamoyl group having a C₁₋₄ alkyl group, N-(2-methacryloyloxyethyl)carbamoyloxy group or N-(2-acryloyloxyethyl)carbamoyloxy group.)

n11 represents an integer of 3 to 6, and is preferably 4.

Z¹² represents —CO— or —CO—C═C—, and preferably represents —CO—.

R¹² represents a hydrogen atom, C₁₋₄ straight-chain alkyl group, C₁ or C₂ straight-chain alkoxy group, phenyl group, aryloxy group, vinyl group, acryloylamino group, methacryloylamino group, N-aryloxycarbamoyl group, N-alkyloxycarbamoyl group having a C₁₋₄ alkyl group, N-(2-methacryloyloxyethyl) carbamoyloxy group or N-(2-acryloyloxyethyl) carbamoyloxy group, preferably represents a C₁₋₄ straight-chain alkyl group, phenyl group, acryloylamino group or methacryloylamino group, and more preferably represents a phenyl group, acryloylamino group or methacryloylamino group.

The C₁₋₄ straight-chain alkyl group preferably has 1 to 3 carbon atoms, and more preferably has 2 carbon atoms.

The C₁ or C₂ straight-chain alkoxy group preferably has one carbon atom.

The N-alkyloxycarbamoyl group having a C₁₋₄ alkyl group preferably has 2 to 4 carbon atoms.

Specific examples of the polymerizable liquid crystal compound represented by the formula (1) will be shown below, without limiting this invention.

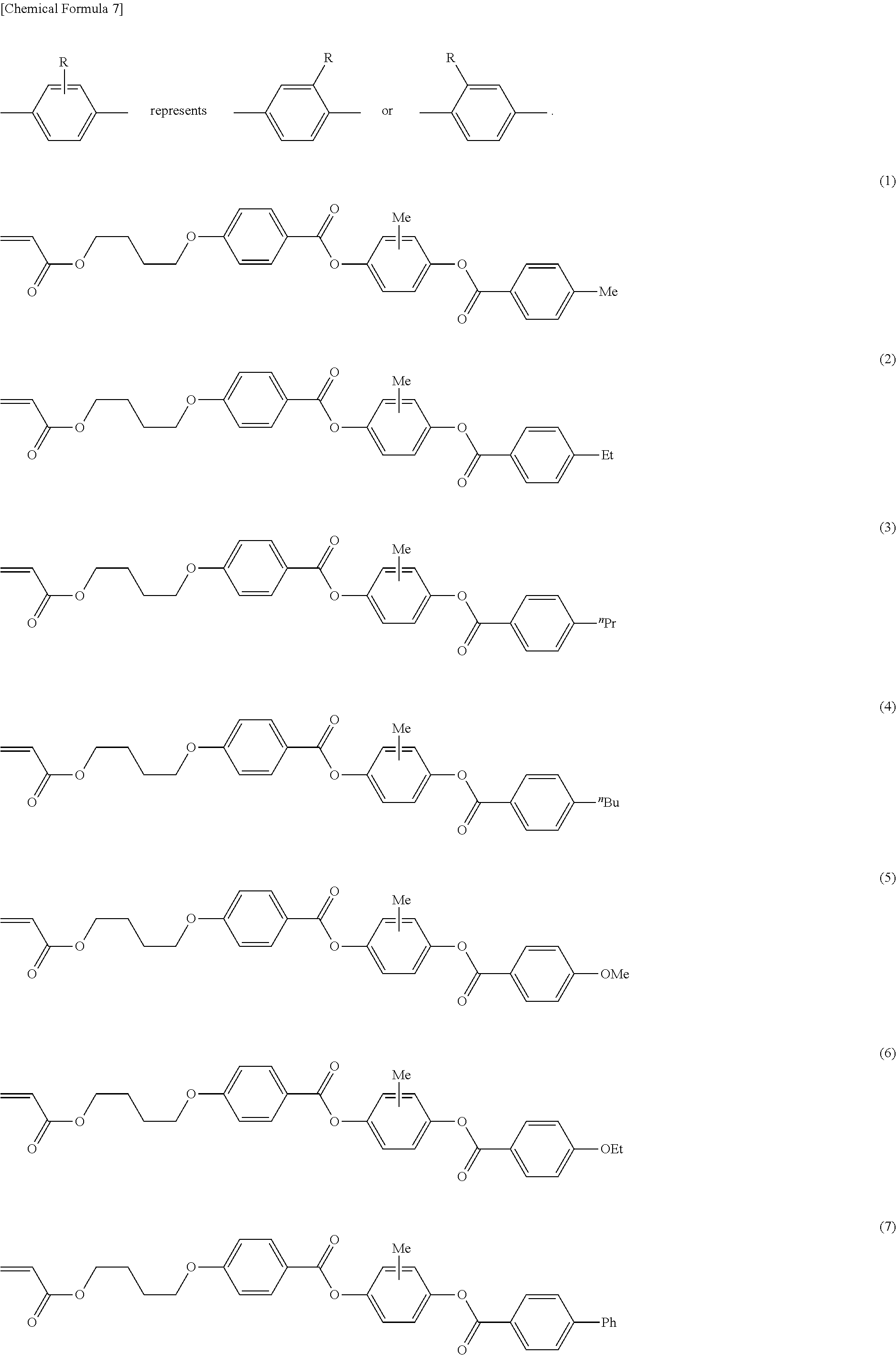

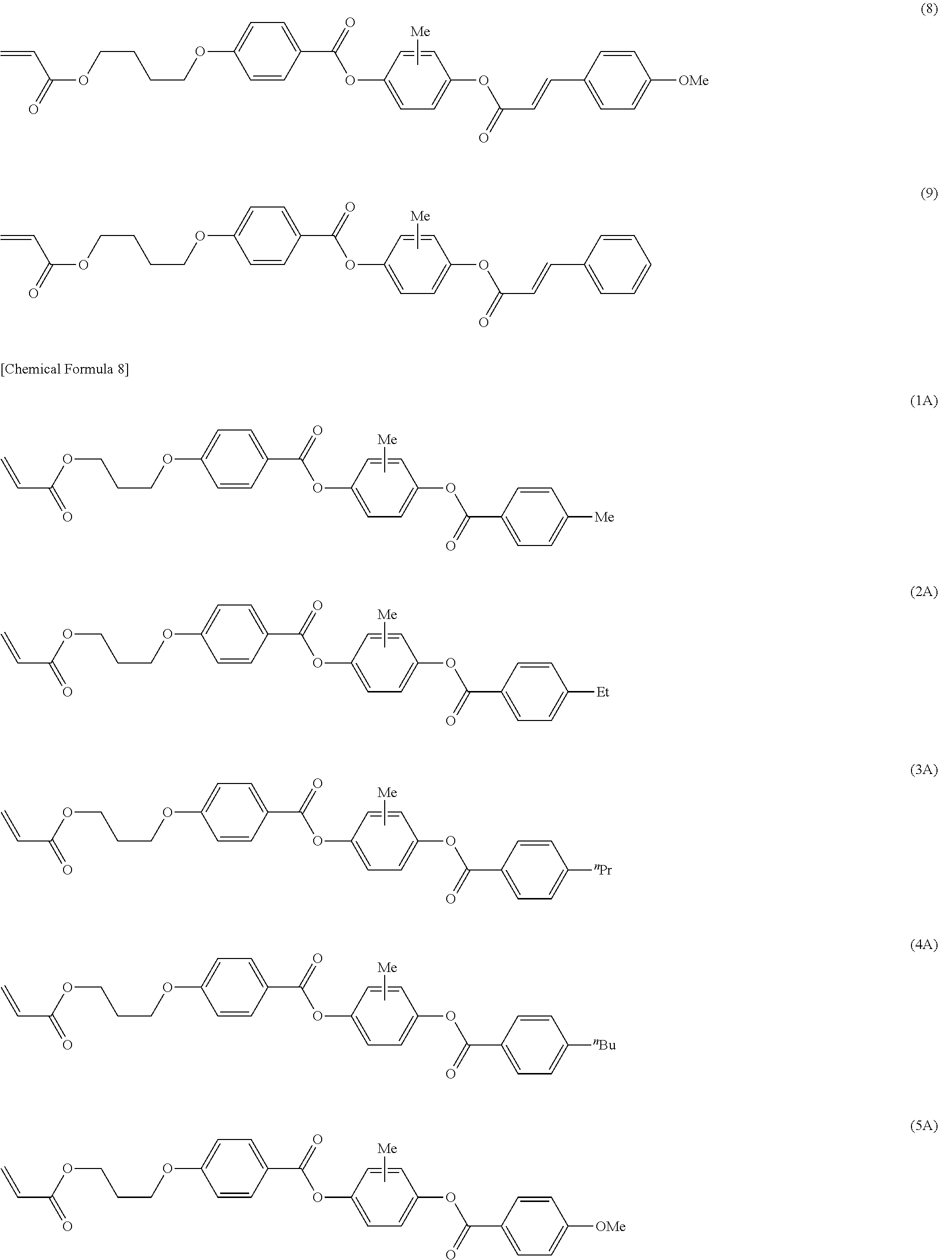

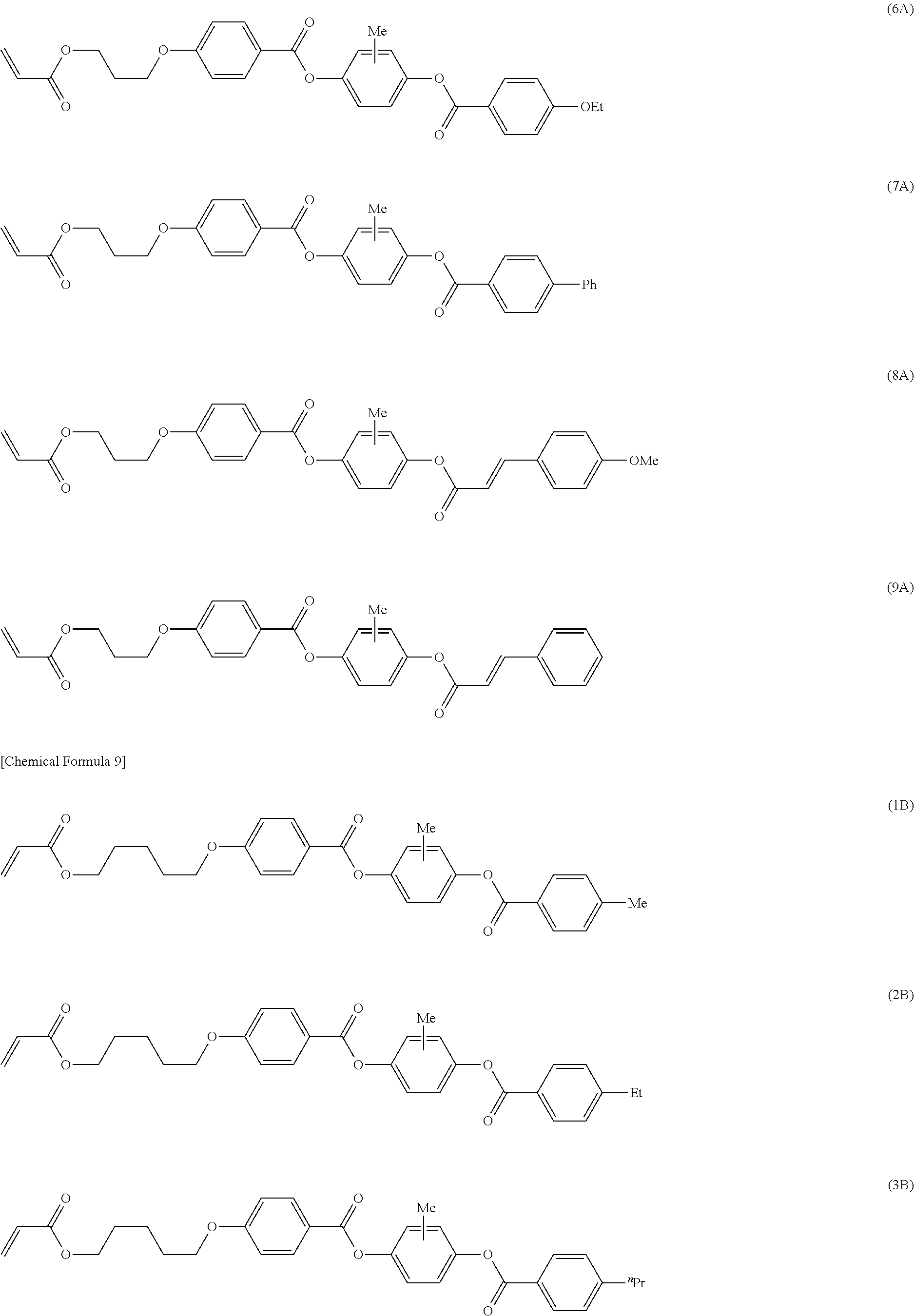

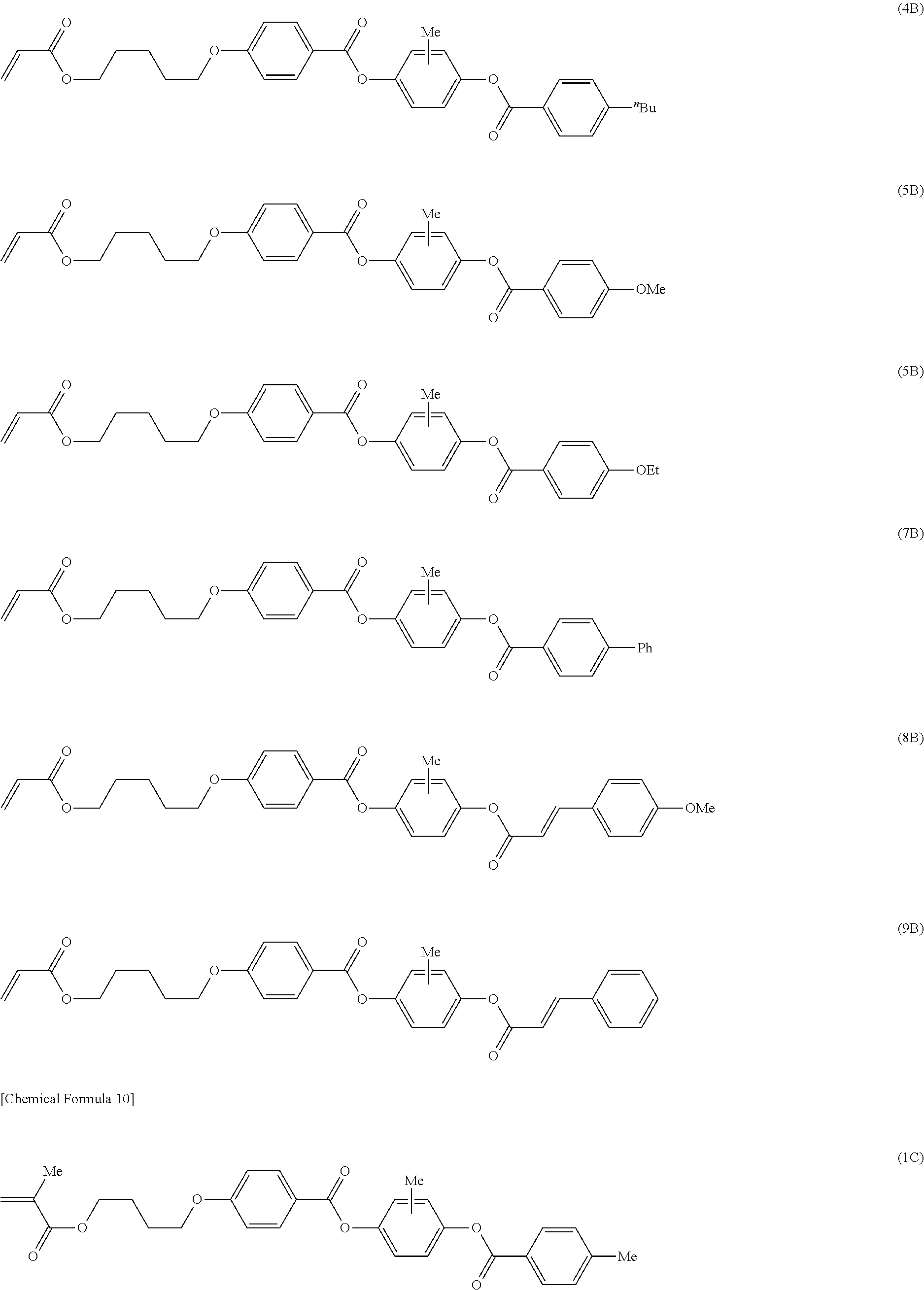

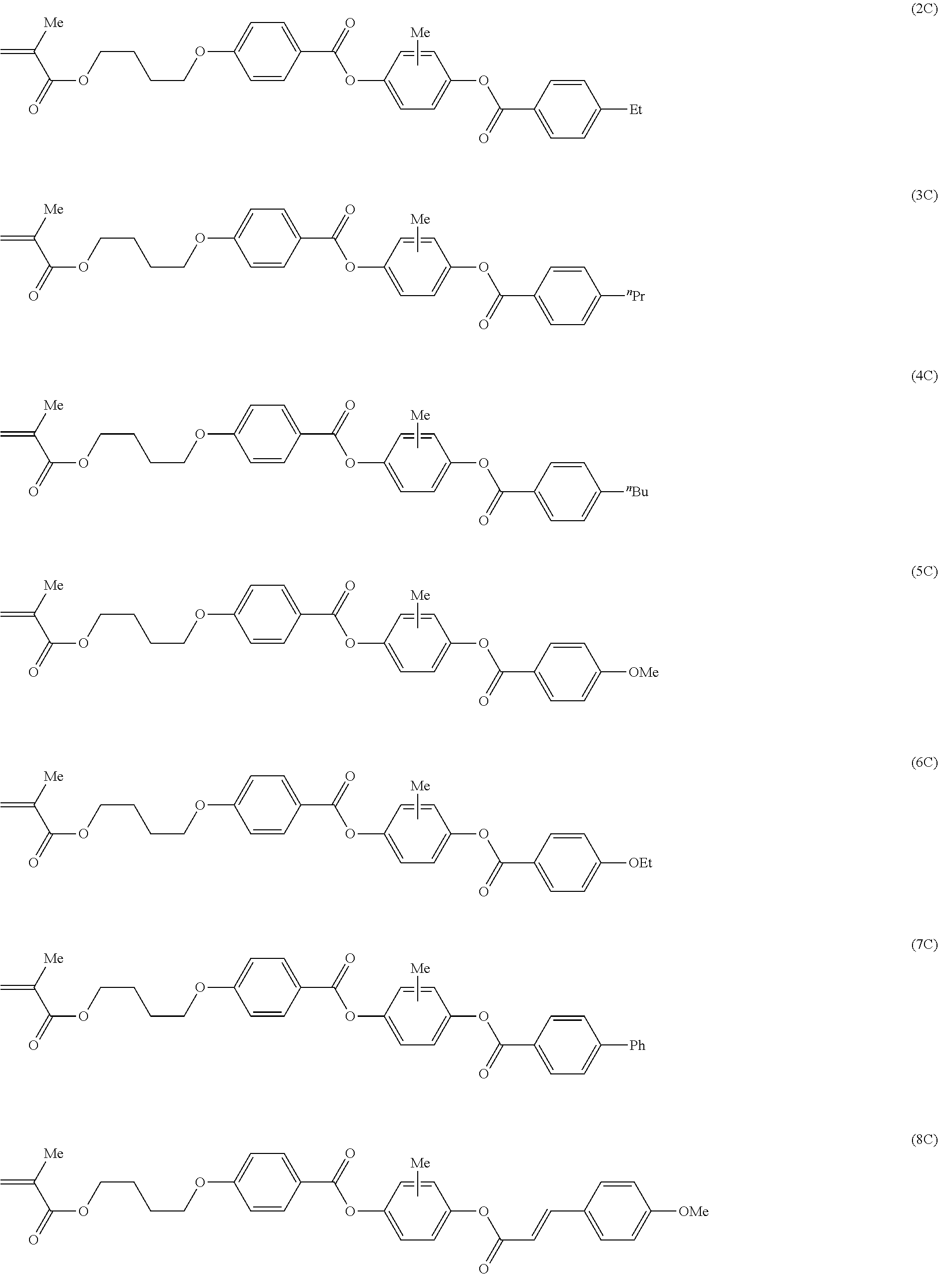

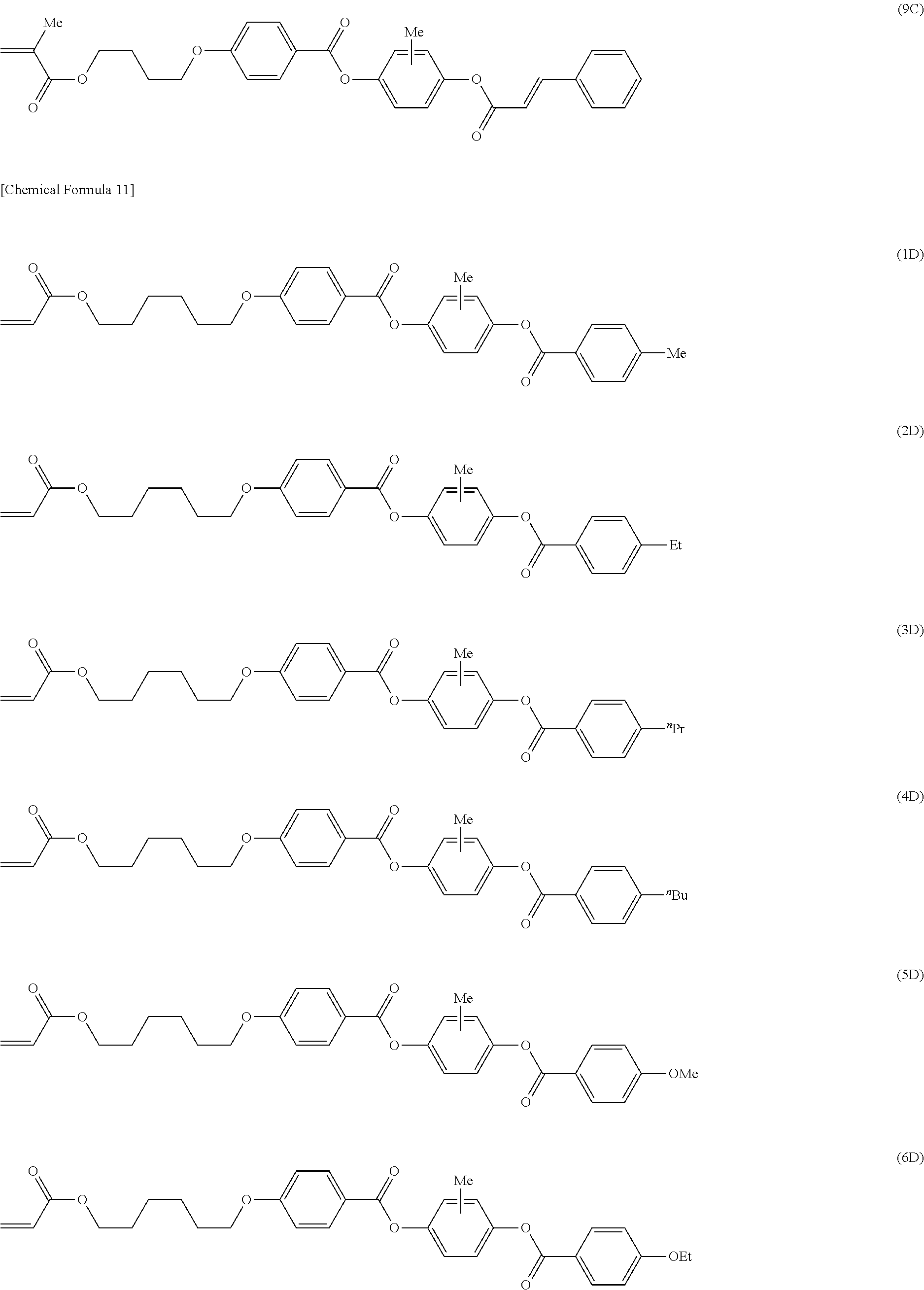

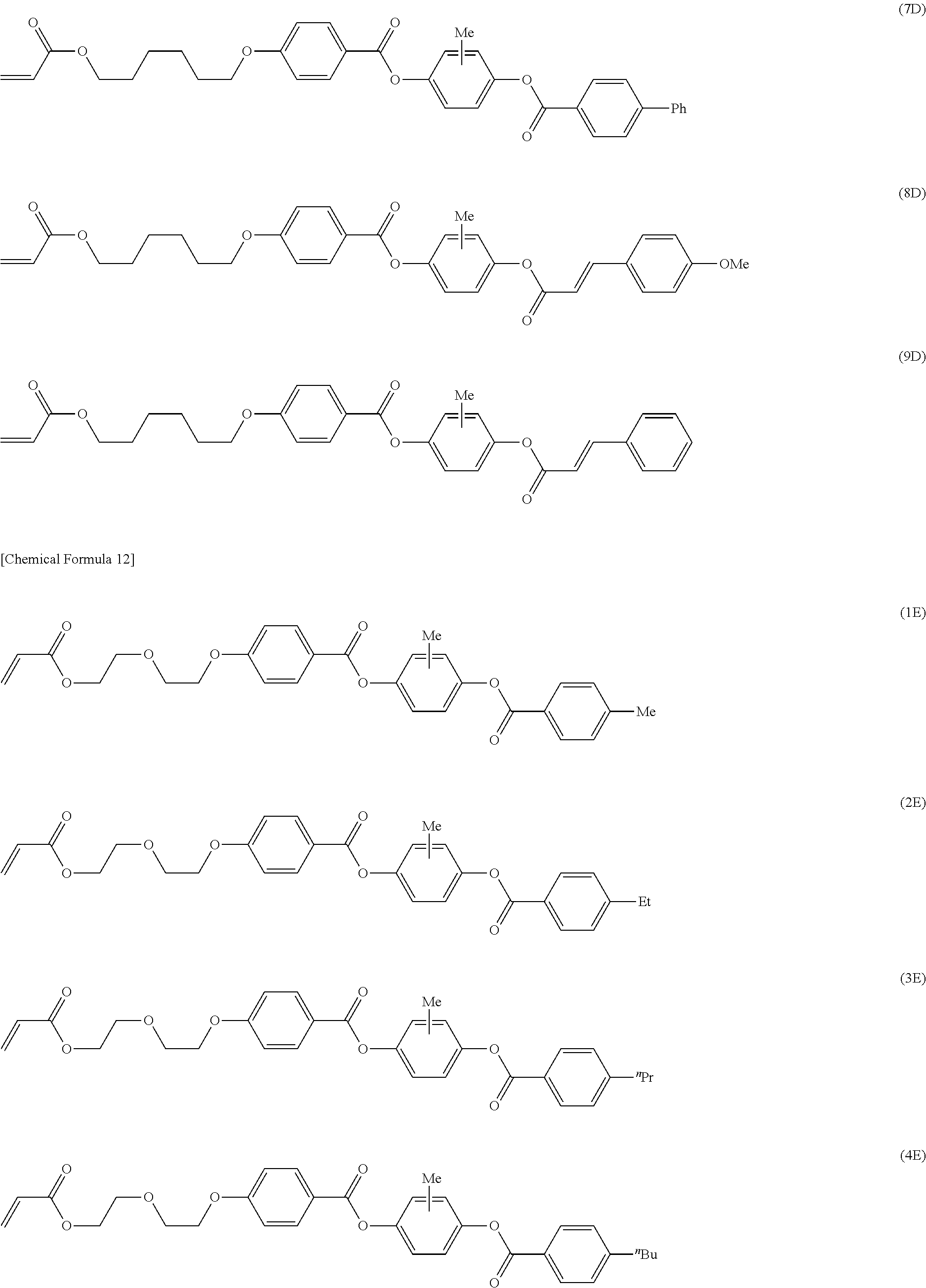

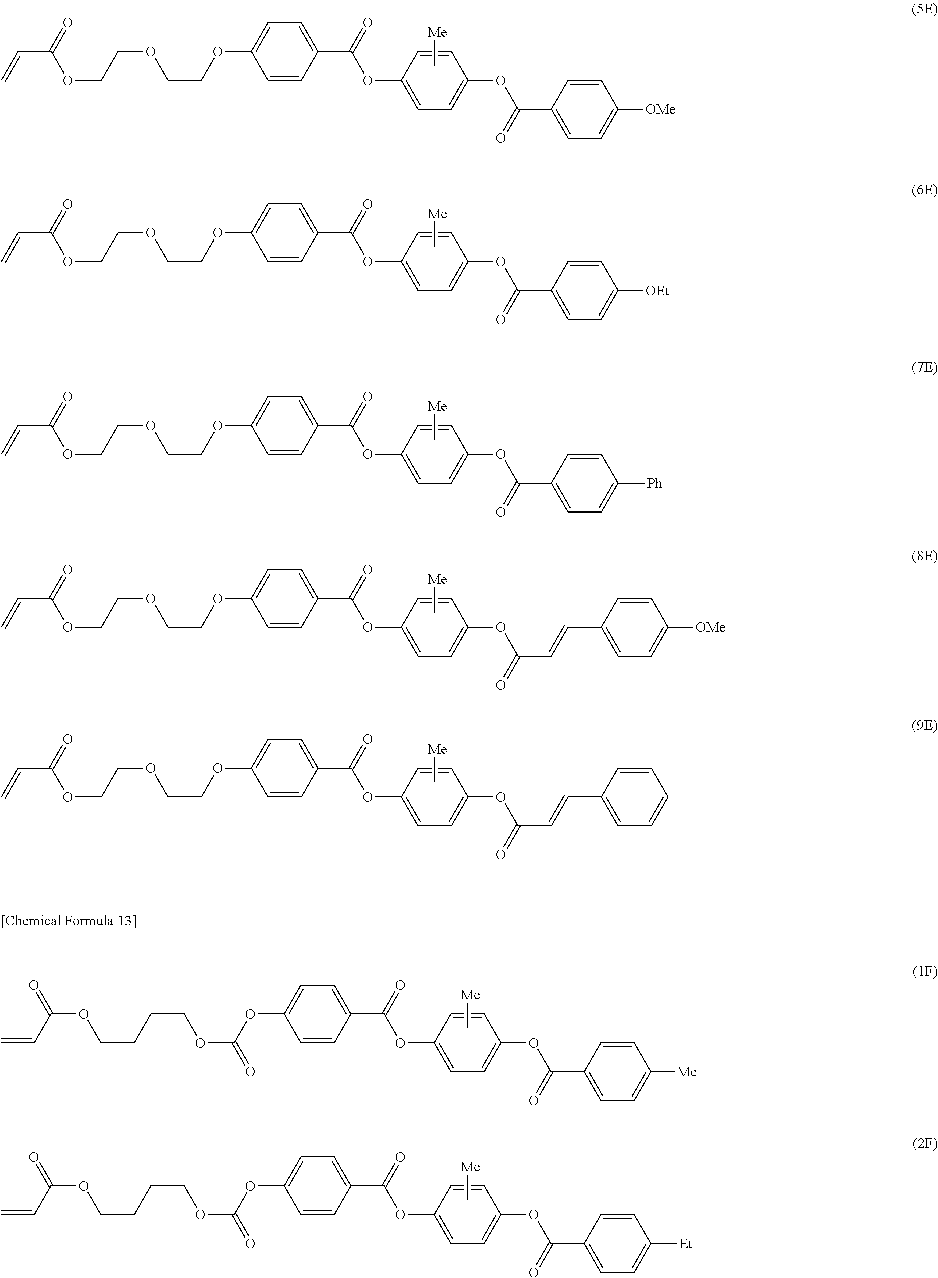

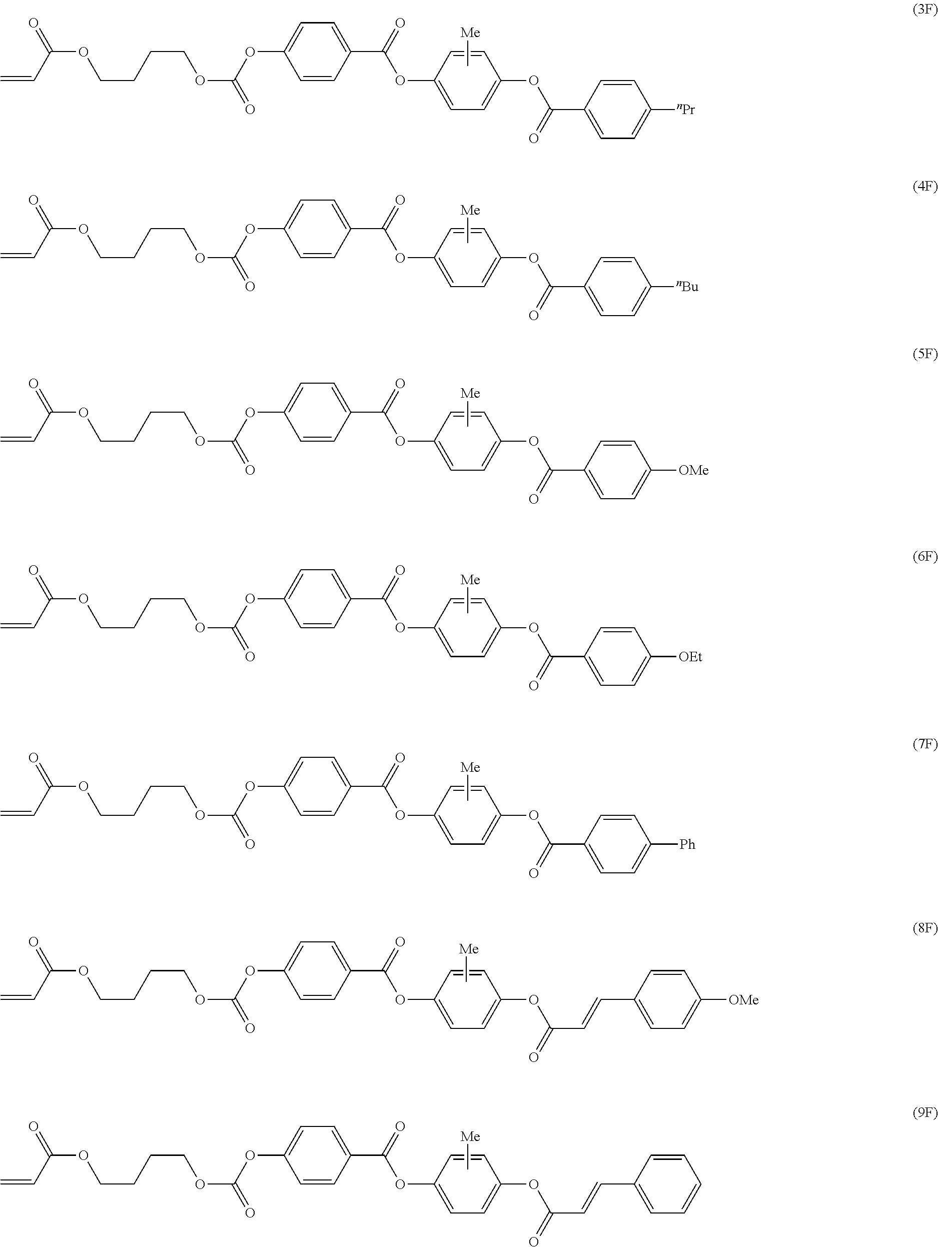

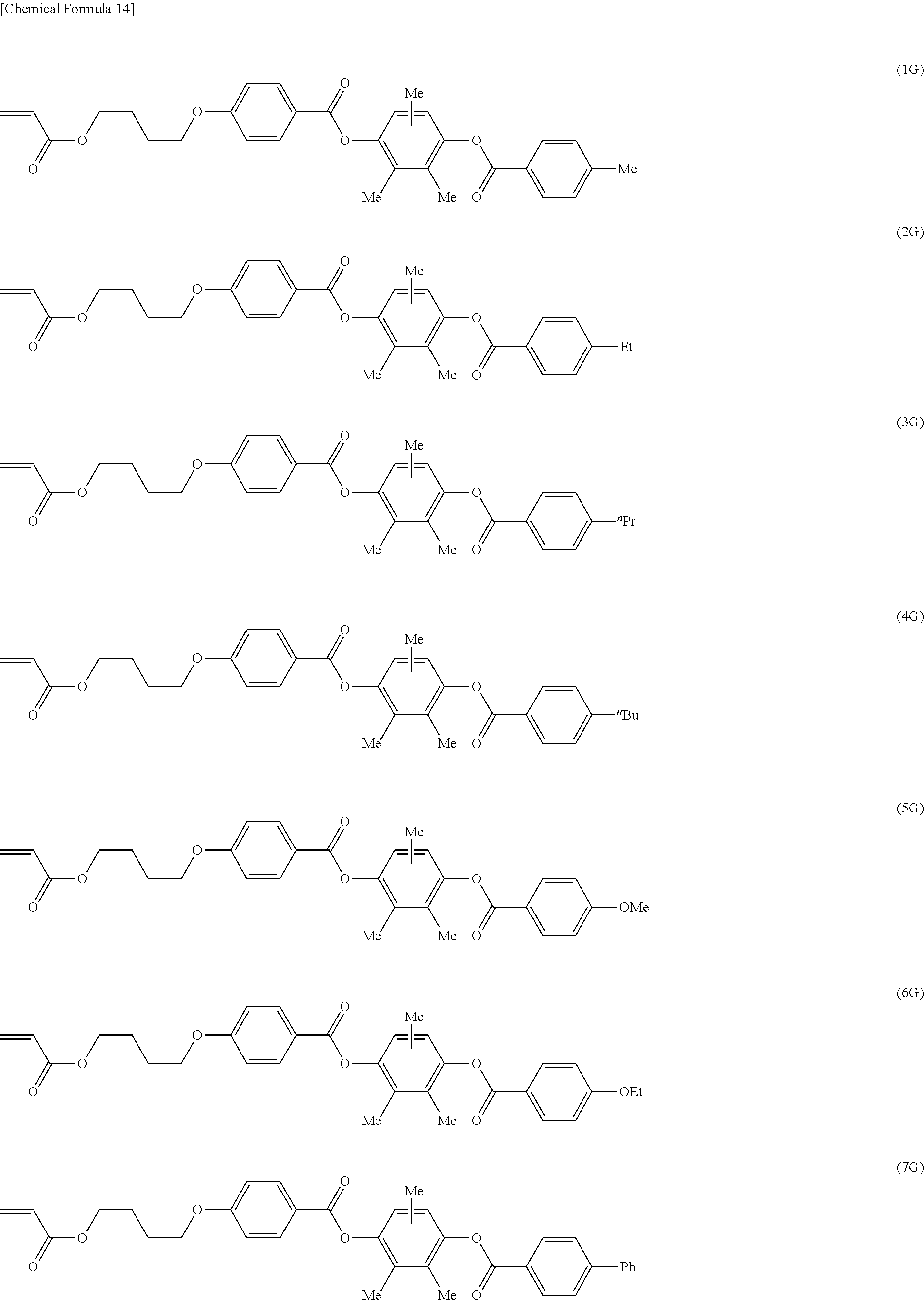

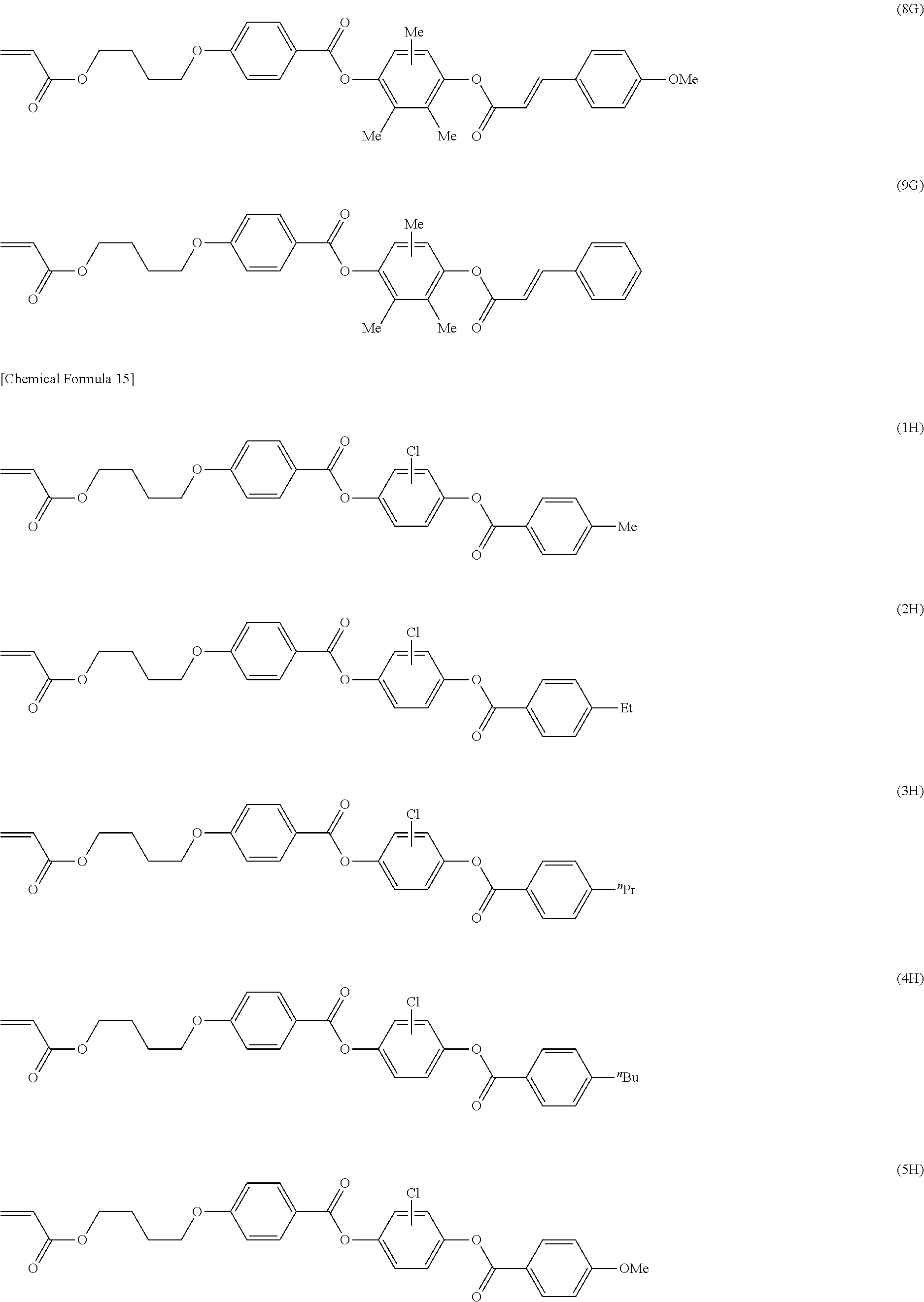

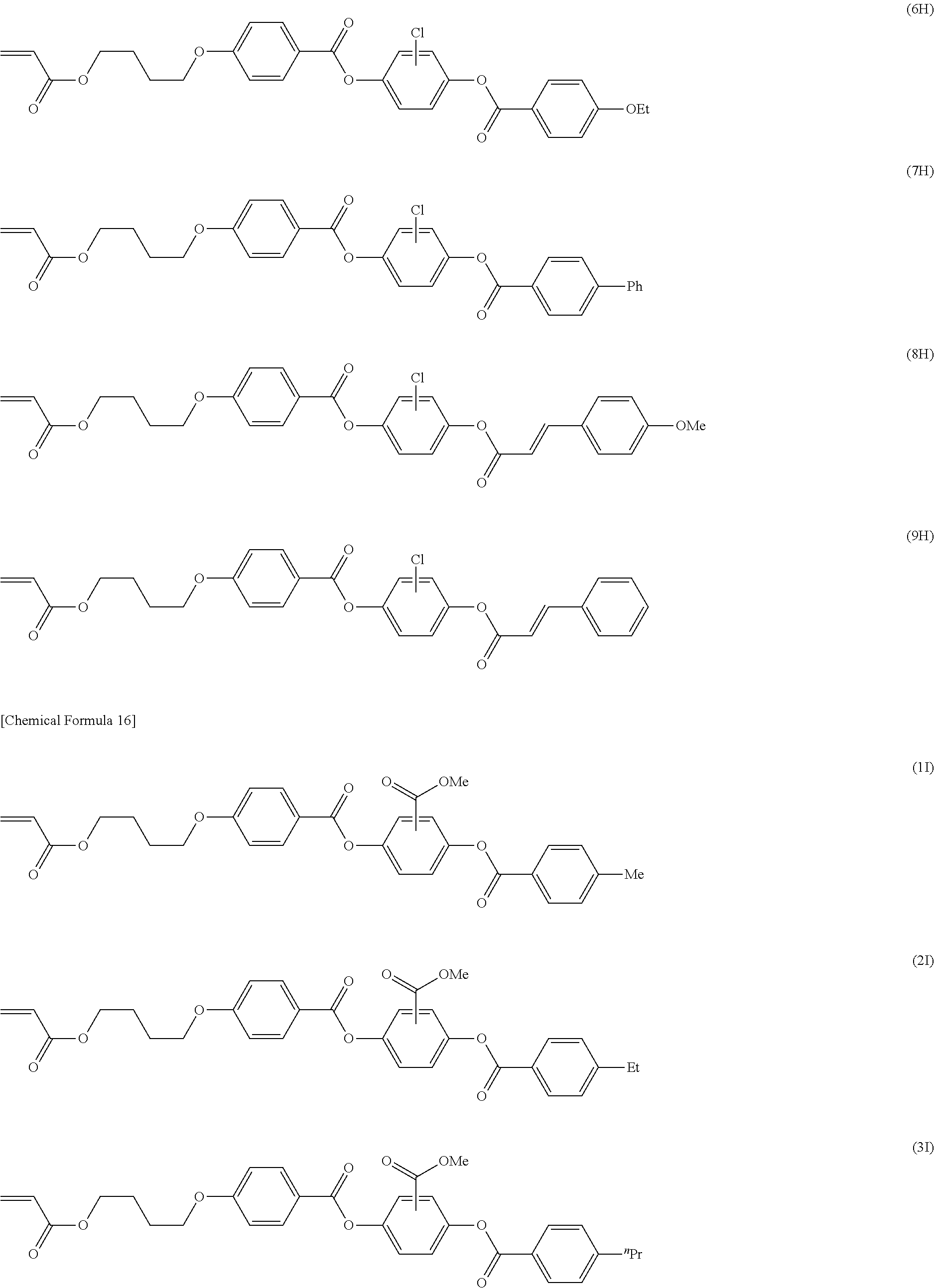

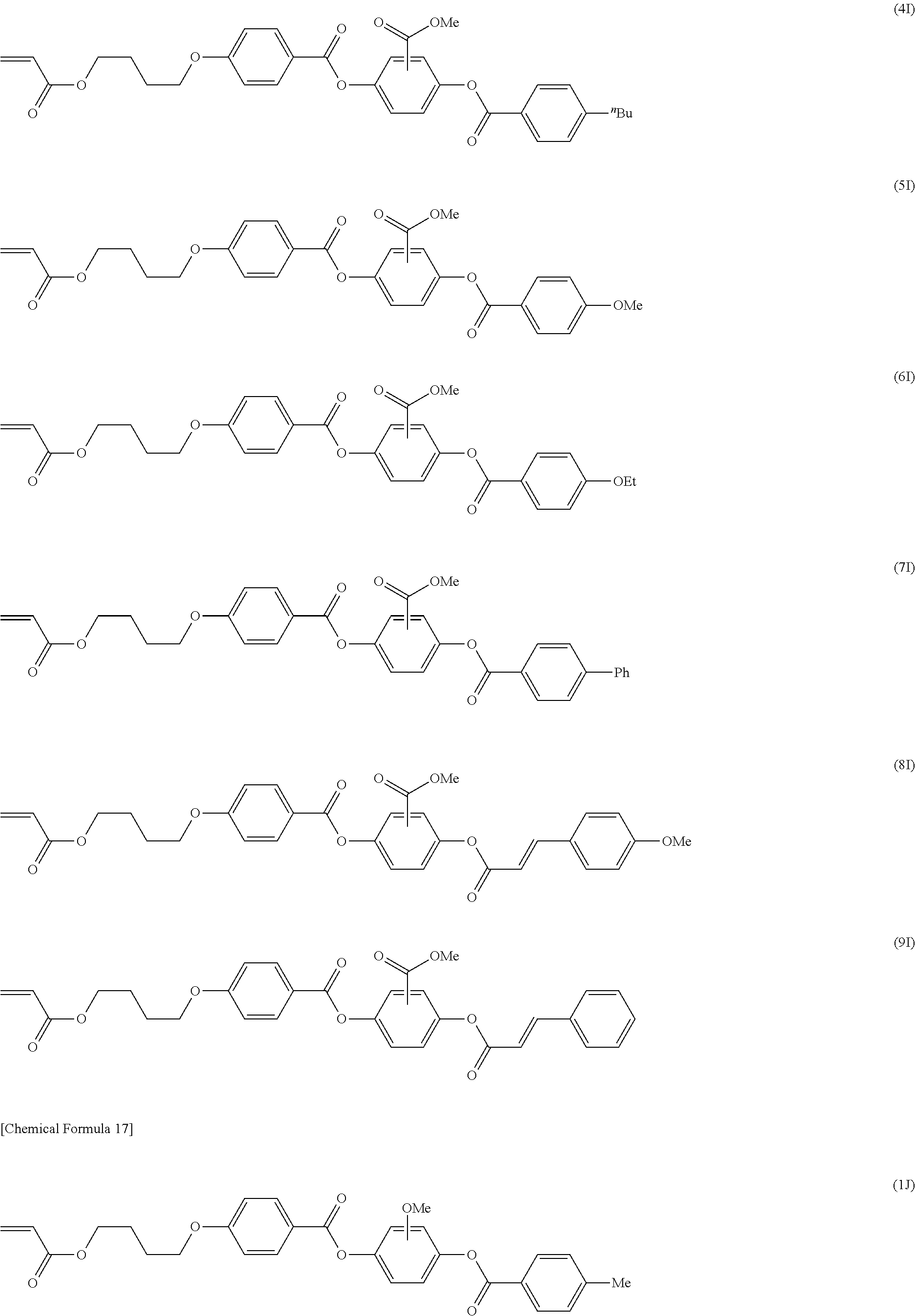

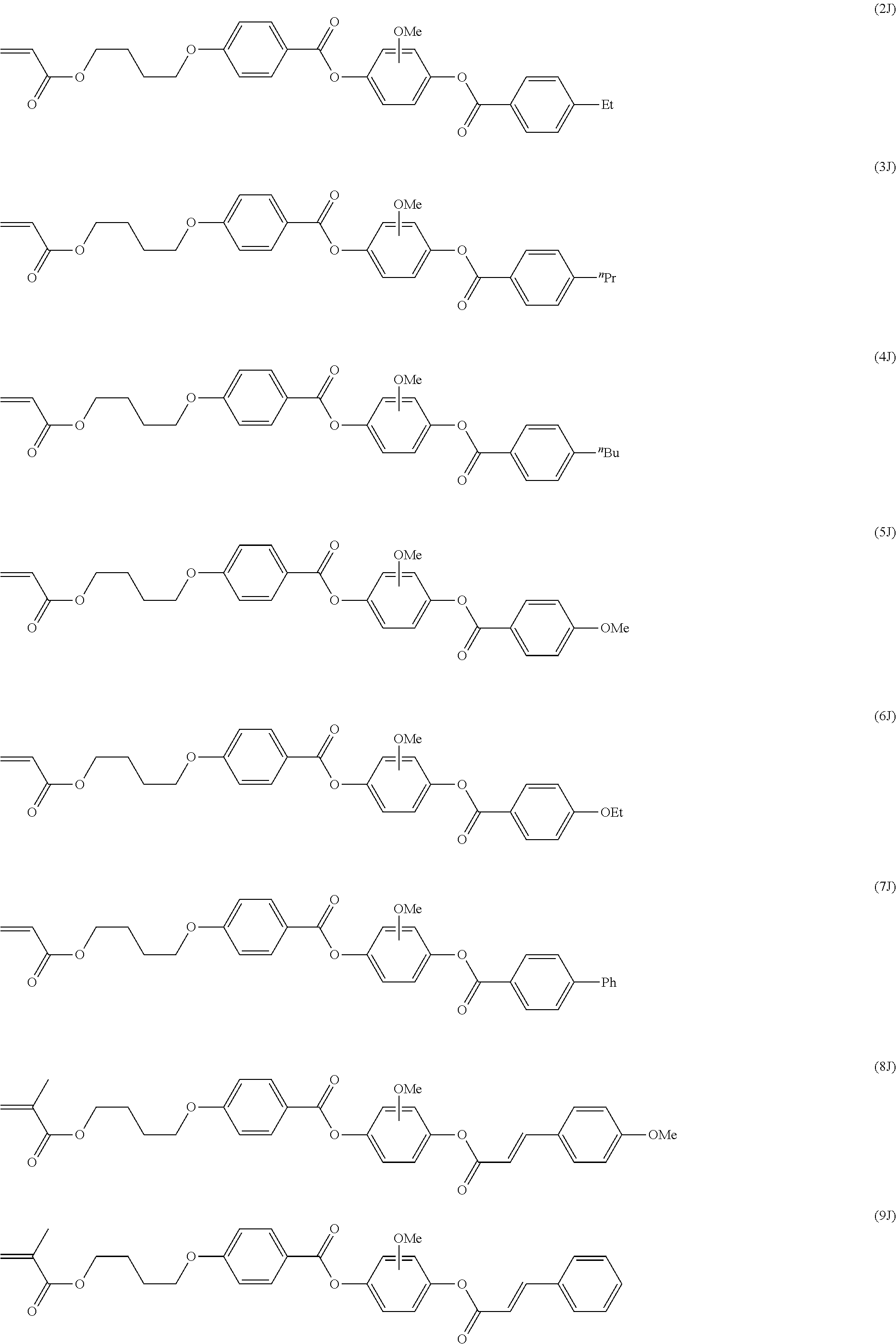

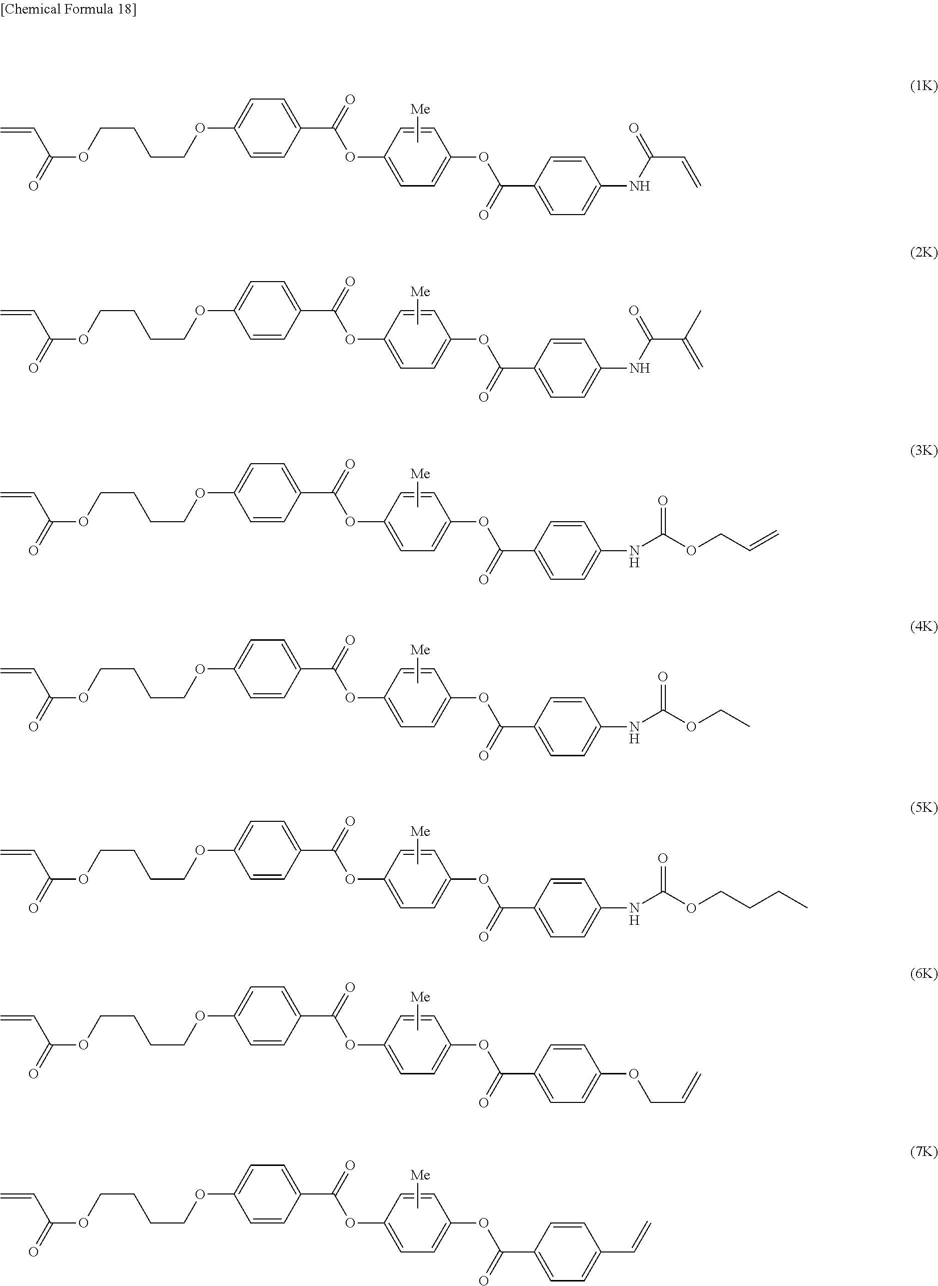

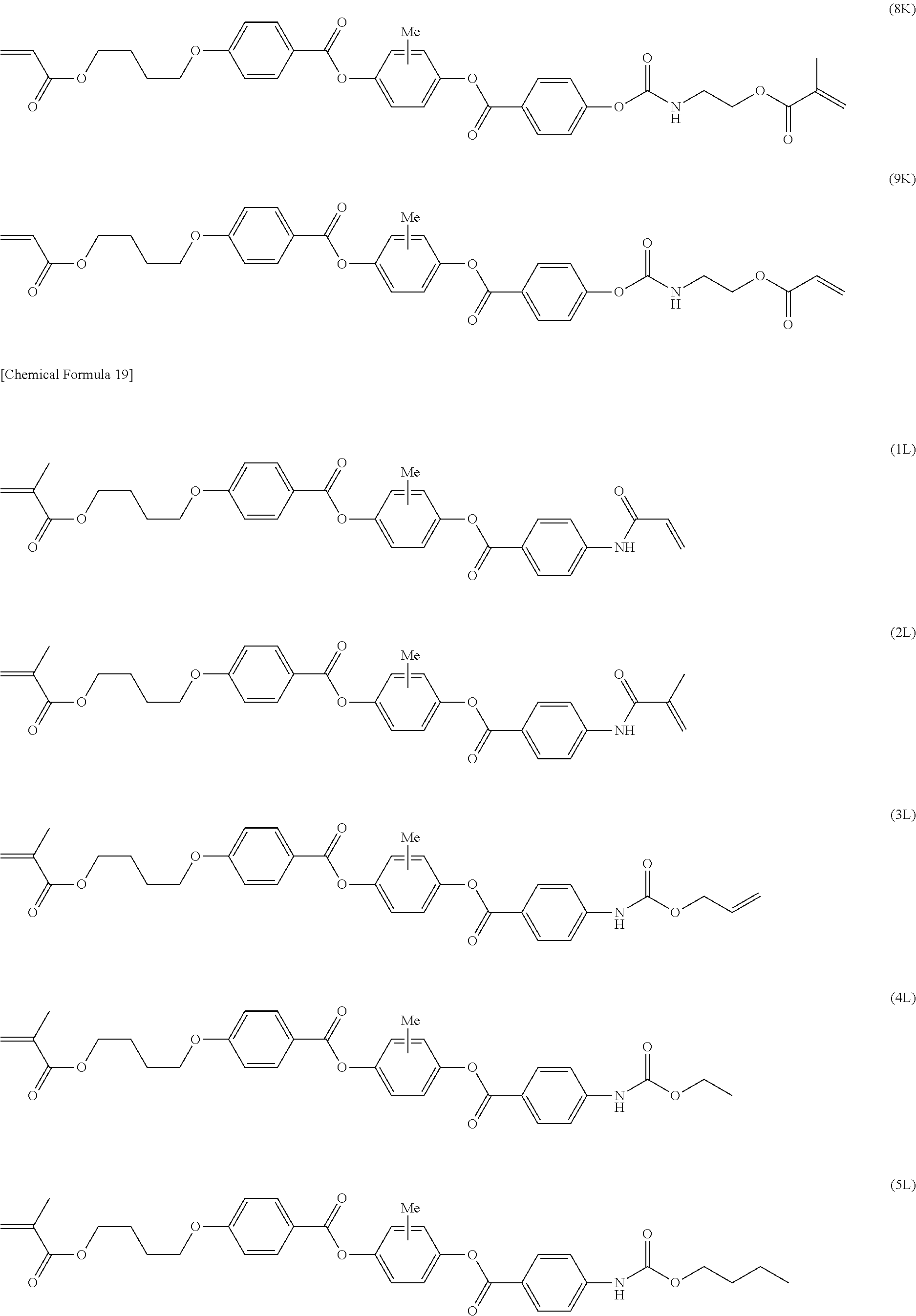

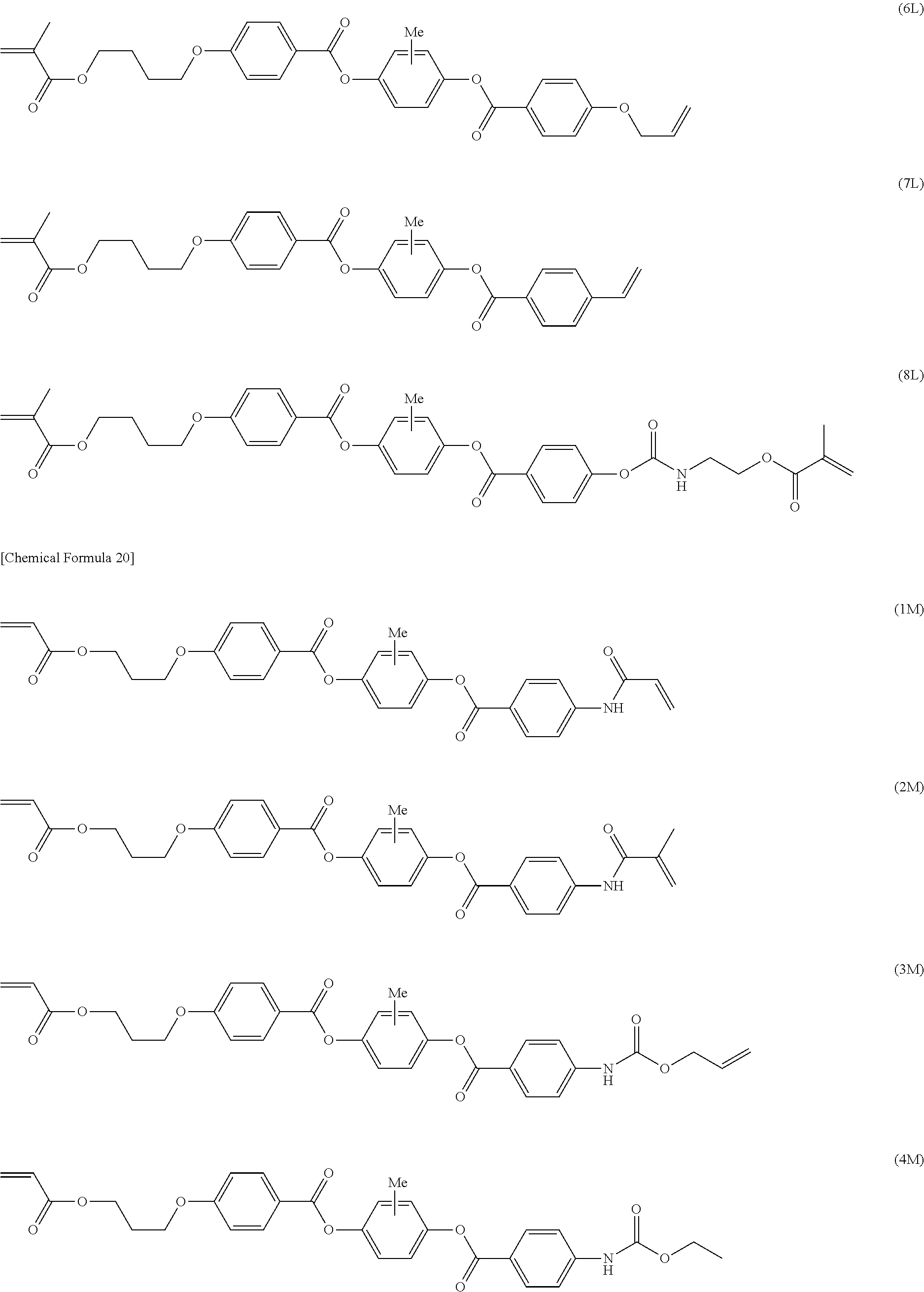

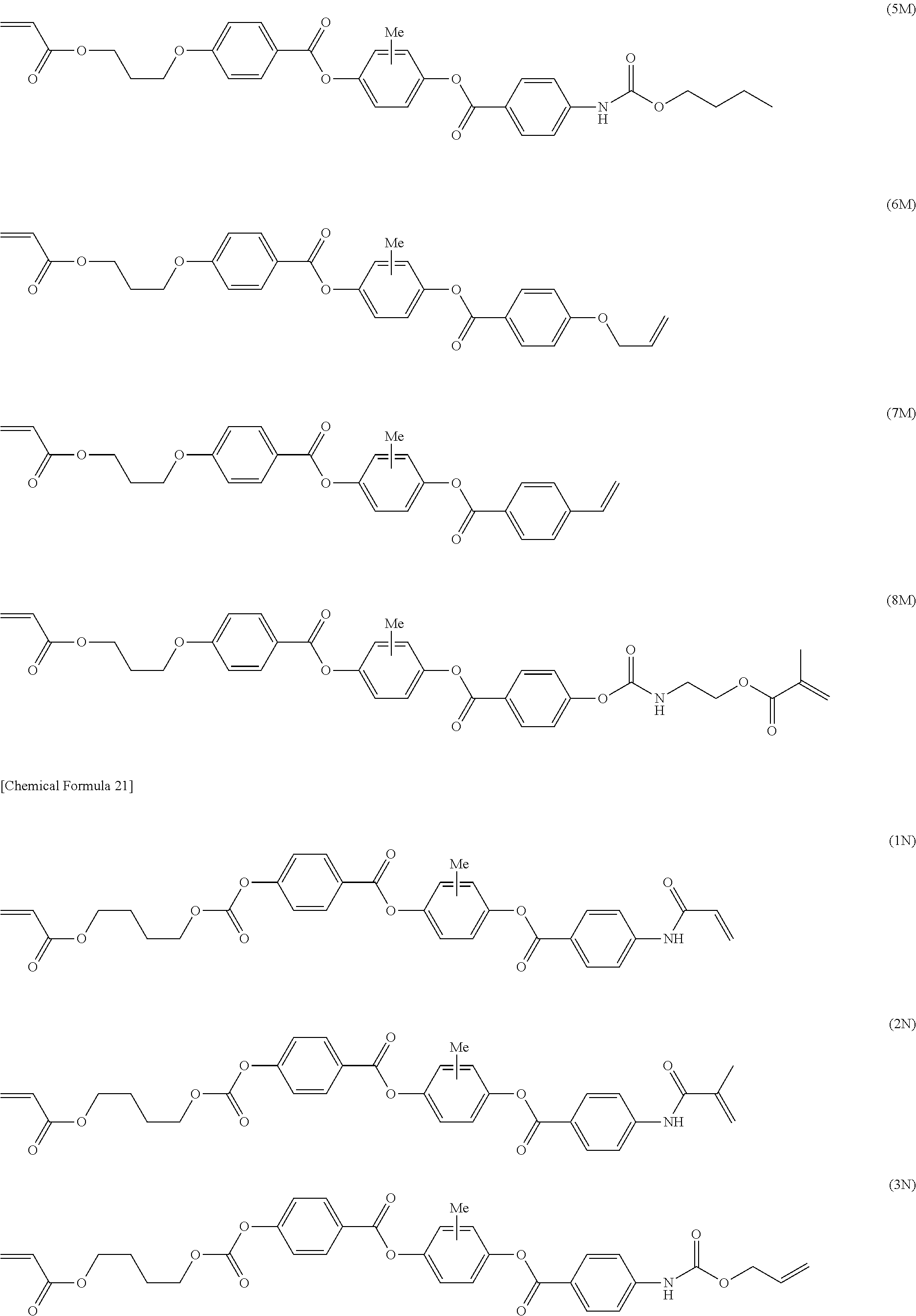

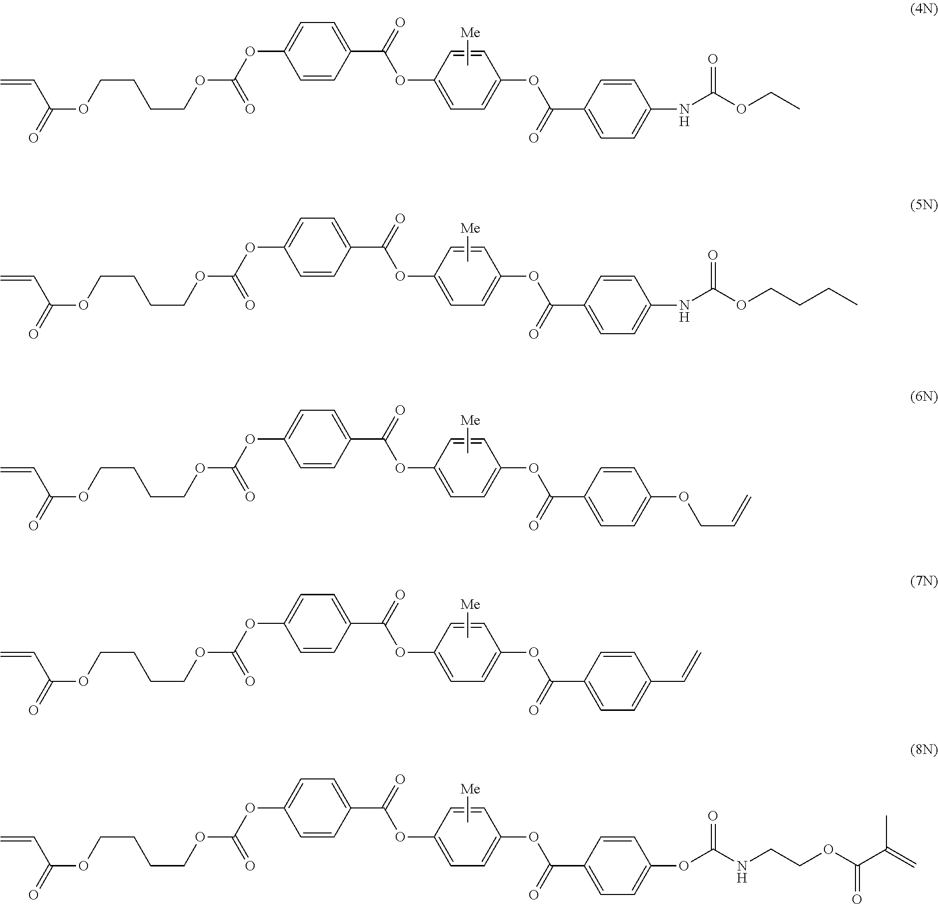

The polymerizable liquid crystal compound represented by the formula (1) above may be manufactured by the methods described in Japanese Translation of PCT International Application Publication No. JP-T2-2002-536529, Molecular Crystals and Liquid Crystals (2010), 530 169-174 and so forth, without special limitation.

### [Liquid Crystal Composition]

The liquid crystal composition of this invention characteristically contains at least one kind of polymerizable liquid crystal compound represented by the formula (1) below, and at least one kind of polymerizable liquid crystal compound represented by the formula (3) below:

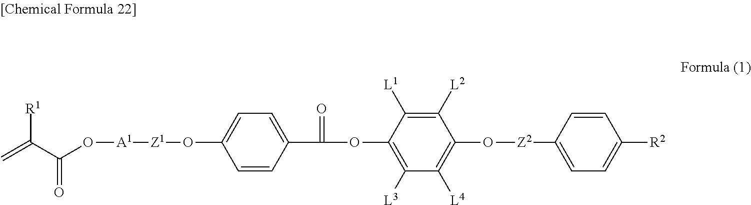

(in the formula (1), A¹ represents a C₂₋₁₈ methylene group, one CH₂ or two or more non-adjacent (CH₂)s in the methylene group may be substituted by —O—;
Z¹ represents —CO—, —O—CO— or single bond;
Z² represents —CO— or —CO—C═C—;
R¹ represents a hydrogen atom or methyl group;
R² represents hydrogen, C₁₋₄ straight-chain alkyl group, C₁ or C₂ straight-chain alkoxy group, phenyl group, aryloxy group, vinyl group, acryloylamino group, methacryloylamino group, N-aryloxycarbamoyl group, N-alkyloxycarbamoyl group having a C₁₋₄ alkyl group, N-(2-methacryloyloxyethyl)carbamoyloxy group or N-(2-acryloyloxyethyl)carbamoyloxy group;
each of L¹, L², L³ and L⁴ independently represents a C₁₋₄ alkyl group, C₁₋₄ alkoxy group, C₂₋₅ alkoxycarbonyl group, C₂₋₄ acyl group, halogen atom or hydrogen atom, and at least one of L¹, L², L³ and L⁴ represents a substituent other than hydrogen atom.)

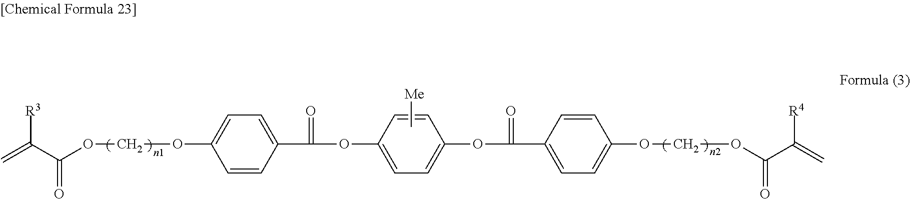

(in the formula (3),
each of n1 and n2 independently represents an integer of 3 to 6;
and
each of R³ and R⁴ independently represents a hydrogen atom or methyl group.)

### (Polymerizable Liquid Crystal Compound Represented by Formula (1))

The liquid crystal composition of this invention contains at least one polymerizable liquid crystal compound represented by the formula (1) above, that is, the polymerizable liquid crystal compound of this invention.

The preferable ranges relevant to the polymerizable liquid crystal compound, represented by the formula (1) above and used for the liquid crystal composition of this invention, are the same as the preferable ranges relevant to the formula (1) and formula (2) above, in the explanation of the polymerizable liquid crystal compound of this invention.

### (Polymerizable Liquid Crystal Compound Represented by Formula (3))

The liquid crystal composition of this invention contains at least one of the polymerizable liquid crystal compounds represented by the formula (3) below:

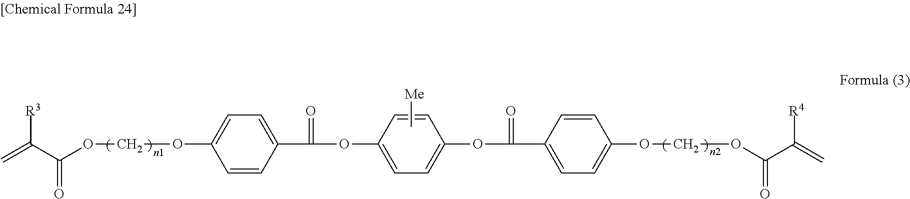

In the formula (3), each of n1 and n2 independently represents an integer of 3 to 6, wherein each of n1 and n2 is preferably 4.

In the formula (3), each of R³ and R⁴ independently represents a hydrogen atom or methyl group, and each of R³ and R⁴ preferably represents a hydrogen atom.

Specific examples of the polymerizable liquid crystal compound represented by the formula (3) will be shown below, without limiting this invention.

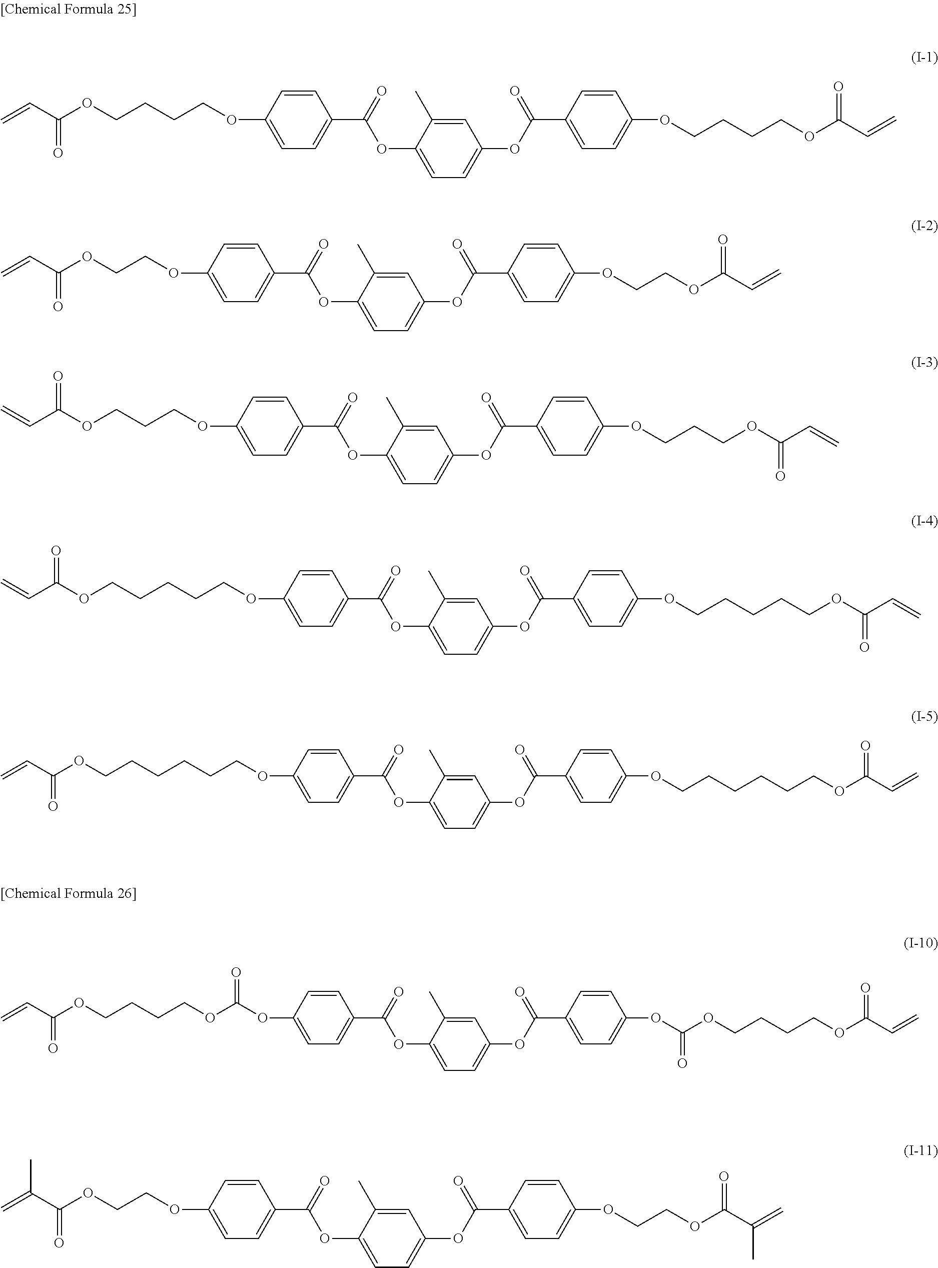

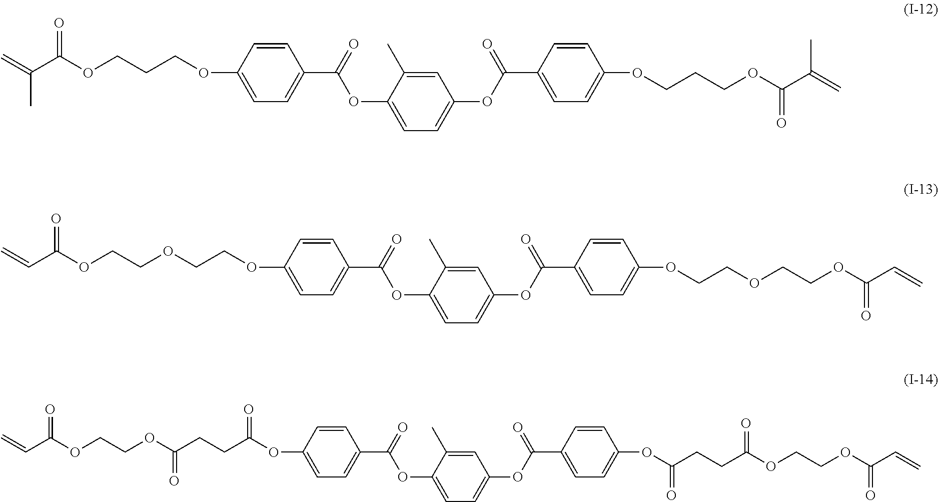

The polymerizable liquid crystal compound represented by the formula (3) above may be manufactured by the methods described in JP-A-2009-184975 and so forth, without special limitation.

### (Compositional Ratio of Polymerizable Liquid Crystal Compounds)

The liquid crystal composition of this invention preferably contains, relative to the polymerizable liquid crystal compound represented by the formula (3) above, 3 to 50% by mass, more preferably 5 to 40% by mass, and particularly 10 to 30% by mass of the polymerizable liquid crystal compound represented by the formula (1) above.

### (Characteristics of the Liquid Crystal Composition)

The liquid crystal composition of the present invention preferably has a nematic-Iso phase transition temperature of 80 to 160° C., and more preferably 90 to 150° C.

### [Polymer Material, Film Configuration]

The polymer material and the film of the present invention each has the polymerizable liquid crystal compound or the optically anisotropic layer obtained by fixing alignment (for example, horizontal alignment, vertical alignment, cholesteric alignment, hybrid alignment, etc.) of the liquid crystal compounds of the liquid crystal composition of the present invention, and has an optical anisotropy. The optically anisotropic layer may be have two or more optically anisotropic layers. The film is usable as an optical compensation film, ½ wavelength film, ¼ wavelength film or phase difference film of liquid crystal display devices based on TN mode, IPS mode and so forth, and as a reflection film making use of selective reflection ascribable to the cholesteric alignment. More preferably the film of the present invention is a film in which the optically anisotropic layer obtainable by fixing a cholesteric alignment of the liquid crystal compounds, and a film obtainable by fixing a cholesteric alignment of the polymerizable liquid crystal compounds of the present invention or the liquid crystal compounds of the liquid crystal composition of the present invention.

Therefore, the liquid crystal composition of the present invention, it is preferable to contain various additives, depending on the application. Following, describing the additive.

### (Other Additives)

The liquid crystal composition of the present invention when used, for example, as a reflection film making use of selective reflection ascribable to the cholesteric alignment, may contain not only the polymerizable liquid crystal, but also optionally contain solvent, compound having chiral carbon atom, polymerizable initiator (described later), and other additives (for example, cellulosic ester).

### Optically Active Compound (Chiral Agent):

The liquid crystal composition may show a cholesteric liquid crystal phase, and for this purpose, preferably contains an optically active compound. Note that if the rod-like liquid crystal compound has a chiral carbon atom, it may sometimes be possible to form the cholesteric liquid crystal phase in a stable manner, without adding the optically active compound. The optically active compound is selectable from publicly known various chiral agents (for example, those described in “Ekisho Debaisu Handobukku (Handbook of Liquid Crystal Devices)”, Chapter 3, Section 4-3, “TN, STN-yo Kairaru-zai (Ciral Agent for TN and STN)”, p. 199, edited by the 142nd Committee of Japan Society for Promoting Science, 1989). While the optically active compound generally has a chiral carbon atom, also axially chiral compound or planar chiral compound having no chiral carbon atom is usable as the chiral agent. Examples of the axially chiral compound and the planar chiral compound include binaphthyl, helicene, paracyclophane, and derivatives of them. The optically active compound (chiral agent) may have a polymerizable group. If the optically active compound has a polymerizable group, and also the rod-like liquid crystal compound used in combination has a polymerizable group, it is now possible to form a polymer having a repeating unit derived from the rod-like liquid crystal compound and a repeating unit derived from the optically active compound, by polymerization reaction between the polymerizable optically active compound and the polymerizable rod-like liquid crystal compound. In this embodiment, the polymerizable group possessed by the polymerizable optically active compound is preferably the same species as the polymerizable group possessed by the polymerizable rod-like liquid crystal compound. Accordingly, also the polymerizable group of the optically active compound is preferably an unsaturated polymerizable group, epoxy group or aziridinyl group, more preferably an unsaturated polymerizable group, and particularly an ethylenic unsaturated polymerizable group.

The optically active compound may also be a liquid crystal compound.

The amount of consumption of the optically active compound in the liquid crystal composition is preferably 1 to 30 mol % of the liquid crystal compound used in combination. The lesser the amount of use of the optically active compound, the better since the liquid crystallinity is less likely to be adversely affected. Accordingly, the optically active compound used as the chiral agent preferably has a strong twisting power, so that a twisted alignment with a desired helical pitch may be obtained only with a small amount of consumption. Such chiral agent showing a strong twisting power is exemplified, for example, by those described in JP-A-2003-287623, which are preferably applicable to the present invention.

### Polymerization Initiator

The polymerization initiator includes a thermal polymerization initiator and a photo-polymerization initiator, and it is preferable to use a photo-polymerization initiator.

Examples of the photo-polymerization initiator include α-carbonyl compounds (described in the specifications of U.S. Pat. Nos. 2,367,661 and 2,367,670), acyloin ether (described in the specification of U.S. Pat. No. 2,448,828), α-hydrocarbon-substituted aromatic acyloin compound (described in the specification of U.S. Pat. No. 2,722,512), polynuclear quinone compounds (described in the specifications of U.S. Pat. Nos. 3,046,127 and 2,951,758), combination of triarylimidazole dimer and p-aminophenyl ketone (described in the specification of U.S. Pat. No. 3,549,367), acrydine and phenazine compounds (described in the specification of JP-A-S60-105667 and U.S. Pat. No. 4,239,850), oxadiazole compound (described in the specification of U.S. Pat. No. 4,212,970), and acylphosphine oxide compounds (described in JP-B-S63-40799, JP-B-H05-29234, JP-A-H10-95788 and JP-A-H10-29997).

The amount of consumption of the photo-polymerization initiator is preferably 0.01 to 20% by mass of the solid content in the coating liquid, and more preferably 0.5 to 5% by mass.

### (Solvent)

Organic solvent is preferably used for dissolving the liquid crystal composition. Examples of the organic solvent include amides (for example, N,N-dimethylformamide), sulfoxides (for example, dimethyl sulfoxide), heterocyclic compounds (for example, pyridine), hydrocarbons (for example, benzene and hexane), alkyl halides (for example, chloroform and dichloromethane), esters (for example, methyl acetate and butyl acetate), ketones (for example, acetone, methyl ethyl ketone, cyclohexanone), and ethers (for example, tetrahydrofuran and 1,2-dimethoxyethane). Alkyl halides and ketones are preferable. Two or more organic solvents may be used in combination.

When the liquid crystal composition of the present invention is used for the optical compensation film of the liquid crystal display device, the liquid crystal composition may contain alignment controlling agent, surfactant, fluorine-containing polymer and so forth, besides the polymerization initiator and the above-described solvent.

### (Polymerization Initiator)

As the polymerization initiator, for the polymerization reaction acceleration of the liquid crystal composition, it is possible to combine several types of the above-mentioned photo-polymerization initiator. For example, it is may be use one of the photo-polymerization initiator of the two kinds of photo-polymerization initiators as sensitizers.

### (Alignment Controlling Agent)

Alignment controlling agent in the present invention is a compound which is typically added to a coating liquid of the liquid crystal composition of the present invention, unevenly distributes, after coated, to the surface of the layer of the liquid crystal composition, or, on the air interface side, and can therefore control alignment of the liquid crystal composition on the air interface side (air interface alignment agent).

As the alignment controlling agent, for example, low-molecular-weight alignment controlling agent and polymer alignment controlling agent are usable. As for the low-molecular-weight alignment controlling agent, for example, paragraphs [0009] to [0083] of JP-A-2002-20363 and paragraphs [0021] to [0029] of JP-A-2012-211306 may be referred to, the contents of which are incorporated into the present specification. As for the polymer alignment controlling agent, for example, paragraphs [0101] to [0105] of JP-A-2005-97377 may be referred to, the contents of which are incorporated into the present specification.

The amount of consumption of the alignment controlling agent is preferably 0.01 to 10% by mass of the solid content in the coating liquid containing the liquid crystal composition of the present invention, and more preferably 0.5 to 5% by mass.

By using such alignment controlling agent and alignment film, the liquid crystal compounds of the present invention may be brought into the state of homogeneous alignment, characterized by an alignment in parallel to the surface of the layer.

When the onium salt or the like is used as the alignment controlling agent, it now becomes possible to promote the homeotropic alignment, at the interface, of the liquid crystal compounds. As for the onium salt which act as a vertical alignment agent, paragraphs [0052] to [0108] of JP-A-2006-106662 may be referred to, the content of which is incorporated into the present specification.

The amount of consumption of the onium salt is preferably 0.01 to 10% by mass of the solid content in the coating liquid containing the liquid crystal composition of the present invention, and more preferably 0.5 to 5% by mass.

### (Surfactant)

Surfactant is exemplified by publicly known compounds, and particularly by fluorine-containing compounds. As for the surfactant, for example, the compounds described in paragraphs [0028] to [0056] of JP-A-2001-330725, and the compounds described in paragraphs [0199] to [0207] of JP-A-2006-106662 may be referred to, the contents of which are incorporated into the present specification.

The amount of consumption of the surfactant is preferably 0.01 to 10% by mass of the solid content in the coating liquid containing the liquid crystal composition of the present invention, and more preferably 0.5 to 5% by mass.

### (Other Additives Applicable to Optical Compensation Film)

As for other additives applicable to the optical compensation film, for example, the compounds described in paragraphs [0099] to [0101] of JP-A-2005-97377 may be referred to, the content of which is incorporated into the present specification.

The film of the present invention may be formed, for example, by coating the liquid crystal composition of the present invention. A preferable method for forming the film of the present invention is such as coating a composition, which contains at least the liquid crystal composition of the present invention, onto the surface of the support, or onto the surface of the alignment film formed thereon, aligning the liquid crystal composition into a desired state, curing it by polymerization, and fixing the state of alignment of the liquid crystal composition.

The liquid crystal composition may be coated by any of publicly known methods (for example, extrusion coating, direct gravure coating, reverse gravure coating, die coating, bar coating, and spin coating). The liquid crystalline molecules are preferably fixed while keeping the state of alignment. The fixation is preferably carried out by a polymerization reaction involving the polymerizable group introduced into the liquid crystalline molecules.

The polymerization reaction includes thermal polymerization reaction making use of a thermal polymerization initiator, and photo-polymerization reaction making use of a photo-polymerization initiator. The photo-polymerization reaction is preferable.

For photo-irradiation for polymerization of discotic liquid crystalline molecule, ultraviolet radiation is preferably used. Irradiation energy is preferably 20 mJ/cm² to 50 J/cm², and more preferably 100 to 800 mJ/cm². The photo-irradiation may be carried out under heating, for the purpose of accelerating the photo-polymerization reaction.

In the present invention, it is preferable to carry out the the polymerization by irradiation with ultraviolet rays.

The thickness of the optically anisotropic layer composed of the liquid crystal composition is preferably 0.1 to 50 μm, and more preferably 0.5 to 30 μm.

For a particular case where selective reflectivity of the film, having the cholesteric alignment of the liquid crystal compounds fixed therein, is utilized, the thickness is more preferably 1 to 30 μm, and most preferably 2 to 20 μm. The total amount of coating of the compound represented by the formula (1) and the compound represented by the formula (3) in the liquid crystal layer (amount of coating of liquid crystal alignment accelerator) is preferably 0.1 to 500 mg/m², more preferably 0.5 to 450 mg/m², furthermore preferably 0.75 to 400 mg/m², and most preferably 1.0 to 350 mg/m².

On the other hand, when the optically anisotropic layer is used as the optical compensation film (for example, A-plate having a state of homogeneous alignment fixed therein, and C-plate having a state of homeotropic alignment fixed therein), the thickness thereof is preferably 0.1 to 50 μm, and more preferably 0.5 to 30 μm.

The alignment film may be provided by a technique such as rubbing of organic compound (preferably polymer), oblique evaporation of inorganic compound, formation of a layer having micro-grooves, or accumulation of organic compound by the Langmuir-Blodgett process (LB film) (for example, ω-tricosanoic acid, dioctadecylmethylammonium chloride, methyl stearate). Also known is an alignment film which turns to demonstrate the alignment function after exposed to electric field, magnetic field, or photo-irradiation. The alignment film formed by rubbing polymer is particularly preferable. The rubbing process is carried out by unidirectionally rubbing the surface of a polymer layer several times with paper or cloth. Species of the polymer used for the alignment film is determined depending on alignment of the liquid crystalline molecules (in particular, average tilt angle). A polymer (general polymer for forming alignment film), which is unlikely to reduce the surface energy of the alignment film is used for the purpose of horizontally aligning the liquid crystalline molecules (with an average tilt angle of 0 to 50°). A polymer capable of reducing the surface energy of the alignment film is used for the purpose of vertically aligning the liquid crystalline molecules (with an average tilt angle of 50 to 90°). In order to reduce the surface energy of the alignment film, it is preferable to introduce a C₁₀₋₁₀₀ hydrocarbon group to a side chain of the polymer.

Species of the polymer are specifically described in literatures regarding the optical compensation sheet using the liquid crystalline molecules adapted to various types of display mode.

The thickness of the alignment film is preferably 0.01 to 5 μm, and more preferably 0.05 to 1 μm. It is also possible to align, by using the alignment film, the liquid crystalline molecules for the optically anisotropic layer, and then transfer the liquid crystal layer onto a translucent support. The liquid crystalline molecules fixed in the aligned state can keep such aligned state without the alignment film. If the average tilt angle is smaller than 5°, rubbing is no longer necessary, and also the alignment film is no longer necessary. However, for the purpose of improving adhesiveness between the liquid crystalline molecules and the translucent support, it is also recommendable to use an alignment film (described in JP-A-H09-152509) which can form a chemical bond with the liquid crystalline molecule at the interface. When the alignment film is used for the purpose of improving the adhesiveness, rubbing is omissible. When two types of liquid crystal layers are provided on the same side of the translucent support, the liquid crystal layer formed on the translucent support may be allowed to function as an alignment film for the liquid crystal layer formed thereon.

The film of the present invention or an optically anisotropic element having the film of the present invention may have the translucent support. Glass plate or polymer film may be used as the translucent support, wherein the polymer film is preferably used. When stating that “the support is translucent”, it means that the light transmittance is 80% or above. The translucent support generally used is an optically isotropic polymer film. The optical isotropy is preferably represented by an in-plane retardation (Re) of smaller than 10 nm, and more preferably smaller than 5 nm. As for the optically isotropic translucent support, also the thickness direction retardation (Rth) is preferably smaller than 10 nm, and more preferably smaller than 5 nm.

### (Selective Reflection Characteristic)

The film of the present invention, having fixed therein the cholesteric liquid crystal phase of the liquid crystal composition of the present invention, preferably shows a selective reflection characteristic, and more preferably shows a selective reflection characteristic in the infrared wavelength region. The light reflective layer having the cholesteric liquid crystal phase fixed therein is detailed in relation to methods described in JP-A-2011-107178 and JP-A-2011-018037, which are also preferably used in the present invention.

### (Laminate)

The film of the present invention is also preferably configured as a laminate of a plurality of layers each having fixed therein the cholesteric liquid crystal phase of the liquid crystal composition of the present invention. The liquid crystal composition of the present invention is also suitable for lamination, and can therefore form such laminate easily.

### (Optical Compensation Film)

The film of the present invention is also usable as an optical compensation film. For example, the films of the present invention can be used as a positive A plate. Herein, the positive A plate means the uniaxial birefringent layer that the refractive index of the slow axis is greater than the refractive index of the thickness direction. Positive A plate, for example, may be obtained by the horizontal orientation of the liquid crystal composition of the present invention (eg, rod-like liquid crystal).

When the film of the present invention is used as the optical compensation film, optical properties of the optically anisotropic layer in the optical compensation film are determined based on optical properties of a liquid crystal cell, and more specifically based on variation in the display mode. By using the liquid crystal composition of the present invention, it is now possible to manufacture the optically anisotropic layer having various optical properties adaptable to various display modes of the liquid crystal cell.

For example, as for the optically anisotropic layer for TN-mode liquid crystal cell, descriptions in JP-A-H06-214116, U.S. Pat. No. 5,583,679, U.S. Pat. No. 5,646,703 and German Patent No. 3911620A1 may be referred to, the contents of which are incorporated into the present specification. As for the optically anisotropic layer for IPS-mode or FLC-mode liquid crystal cell, descriptions in JP-A-H09-292522 and JP-A-H10-54982 may be referred to, the contents of which are incorporated into the present specification. As for the optically anisotropic layer for OCB-mode or HAN-mode liquid crystal cell, the descriptions in U.S. Pat. No. 5,805,253 and International Patent Application WO96/37804 may be referred to, the contents of which are incorporated into the present specification. As for the optically anisotropic layer for STN-mode liquid crystal cell, the description in JP-A-H09-26572 may be referred to, the content of which is incorporated into the present specification. As for the optically anisotropic layer for VA-mode liquid crystal cell, the description in Japanese Patent JP-B02-2866372 may be referred to, the content of which is incorporated into the present specification.

In particular, in the present invention, the film of this invention is preferably used as the optically anisotropic layer of the IPS-mode liquid crystal cell.

For example, a film having an optically anisotropic layer, in which the liquid crystal compounds of the present invention is in the state of homogeneous alignment, is usable as an A-plate. The A-plate now means a uniaxial birefringent layer characterized by the refractive index in the slow axis direction larger than the refractive index in the thickness direction. When the film of the present invention is the A-plate, only a single optically anisotropic layer will suffice for compensation, if the layer shows an in-plane retardation (Re) of 200 nm to 350 nm at 550 nm.

A film having an optically anisotropic layer, in which the liquid crystal compounds of the present invention is in the state of homeotropic alignment, is usable as a positive C-plate, possibly in combination with a biaxial film or the like. The positive C-plate now means a uniaxial birefringent layer characterized by the refractive index in the thickness direction larger than the in-plane refractive index. The film of the present invention, used as the positive C-plate, preferably has an in-plane retardation (Re) at 550 nm of −10 nm to 10 nm, and a thickness direction retardation (Rth) at 550 nm of −250 to −50 nm, although depending on optical characteristics of the biaxial film to be combined.

### [Polarizing Plate]

The present invention also relates to a polarizing plate having at least the film with the optically anisotropic layer (optical compensation film), and a polarizing film. In the polarizing plate having a polarizing film and a protective film disposed at least on one side thereof, the optically anisotropic layer is usable as such protective film.

Alternatively, in the polarizing plate configured to have the protective films on both sides of the polarizing film, the optically anisotropic layer is also usable as one of these protective films.

The polarizing film includes iodine-containing polarizing film, dye-containing polarizing film using dichroic dye, and polyene-based polarizing film. The iodine-containing polarizing film and the dye-containing polarizing film may be manufactured generally by using polyvinyl alcohol-based film.

Although the thickness of the polarizing film is not specifically limited, the thinner the polarizing film, the more thinner will be the polarizing plate and liquid crystal display device into which it is incorporated. From this point of view, the thickness of the polarizing film is preferably 10 μm or smaller. Since the optical path length in the polarizing film is necessarily longer than the wavelength of light, so that the minimum thickness of the polarizing film is preferably 0.7 μm or larger, substantially 1 μm or larger, and generally 3 μm or larger.

### [Liquid Crystal Display Device]

The present invention also relates to a liquid crystal display device having such polarizing plate. The liquid crystal display device may have any alignment mode, without special limitation, such as TN mode, IPS mode, FLC mode, OCB mode, HAN mode, or VA mode. As for the liquid crystal display device making use of VA mode, the description in paragraphs [0109] to [0129] of JP-A-2005-128503 may be referred to, the content of which is incorporated into the present specification. As for the liquid crystal display device making use of IPS mode, the description in paragraphs [0027] to [0050] of JP-A-2006-106662 may be referred to, the content of which is incorporated into the present specification.

For the liquid crystal display device of the present invention, for example, the A-plate and C-plate described above are usable.

The optically anisotropic layer may be incorporated into the liquid crystal display device, in the form of polarizing plate obtained by bonding with the polarizing film. Alternatively, the optically anisotropic layer may be incorporated as a viewing angle compensation film which is configured by the optically anisotropic layer by itself, or by a laminate combined with other phase difference layer. The other phase difference layer to be combined is selectable, depending on the alignment mode of the liquid crystal cell in need of compensation of viewing angle.

The optically anisotropic layer may be disposed between the liquid crystal cell and the polarizing film on the viewer's side, or between the liquid crystal cell and the polarizing film on the back light side.

In this description, Re(Λ) and Rth(Λ) are retardation (nm) in plane and retardation (nm) along the thickness direction, respectively, at a wavelength of A. Re(Λ) is measured by applying light having a wavelength of Λ nm to a film in the normal direction of the film, using KOBRA 21ADH or WR (by Oji Scientific Instruments). The selection of the measurement wavelength may be conducted according to the manual-exchange of the wavelength-selective-filter or according to the exchange of the measurement value by the program.

When a film to be analyzed is expressed by a monoaxial or biaxial index ellipsoid, Rth(Λ) of the film is calculated as follows.

Rth(Λ) is calculated by KOBRA 21ADH or WR on the basis of the six Re(Λ) values which are measured for incoming light of a wavelength Λ nm in six directions which are decided by a 100 step rotation from 0° to 50° with respect to the normal direction of a sample film using an in-plane slow axis, which is decided by KOBRA 21ADH, as an inclination axis (a rotation axis; defined in an arbitrary in-plane direction if the film has no slow axis in plane), a value of hypothetical mean refractive index, and a value entered as a thickness value of the film.

In the above, when the film to be analyzed has a direction in which the retardation value is zero at a certain inclination angle, around the in-plane slow axis from the normal direction as the rotation axis, then the retardation value at the inclination angle larger than the inclination angle to give a zero retardation is changed to negative data, and then the Rth(Λ) of the film is calculated by KOBRA 21ADH or WR.

Around the slow axis as the inclination angle (rotation angle) of the film (when the film does not have a slow axis, then its rotation axis may be in any in-plane direction of the film), the retardation values are measured in any desired inclined two directions, and based on the data, and the estimated value of the mean refractive index and the inputted film thickness value, Rth may be calculated according to formulae (1) and (2):

$$\begin{matrix}[ Mathematical 1 ] & \\ Re ( θ ) = [ nx - \frac{ny \times nz}{\sqrt{\begin{matrix}{\{ ny sin ( {sin}^{- 1} ( \frac{sin ( - θ )}{nx} ) ) \}}^{2} + \\ {\{ nz cos ( {sin}^{- 1} ( \frac{sin ( - θ )}{nx} ) ) \}}^{2}\end{matrix}}} ] \times \frac{d}{cos \{ {sin}^{- 1} ( \frac{sin ( - θ )}{nx} ) \}} & formulae ( 1 )\end{matrix}$$

Re(Θ) represents a retardation value in the direction inclined by an angle Θ from the normal direction; nx represents a refractive index in the in-plane slow axis direction; ny represents a refractive index in the in-plane direction perpendicular to nx; and nz represents a refractive index in the direction perpendicular to nx and ny. And “d” is a thickness of the film.

When the film to be analyzed is not expressed by a monoaxial or biaxial index ellipsoid, or that is, when the film does not have an optical axis, then Rth(Λ) of the film may be calculated as follows:

Re(Λ) of the film is measured around the slow axis (judged by KOBRA 21ADH or WR) as the in-plane inclination axis (rotation axis), relative to the normal direction of the film from −50° up to +50° at intervals of 10°, in 11 points in all with a light having a wavelength of Λ nm applied in the inclined direction; and based on the thus-measured retardation values, the estimated value of the mean refractive index and the inputted film thickness value, Rth(Λ) of the film may be calculated by KOBRA 21ADH or WR.

In the above-described measurement, the hypothetical value of mean refractive index is available from values listed in catalogues of various optical films in Polymer Handbook (John Wiley & Sons, Inc.). Those having the mean refractive indices unknown can be measured using an Abbe refract meter. Mean refractive indices of some main optical films are listed below:

cellulose acylate (1.48), cycloolefin polymer (1.52), polycarbonate (1.59), polymethylmethacrylate (1.49) and polystyrene (1.59). KOBRA 21ADH or WR calculates nx, ny and nz, upon enter of the hypothetical values of these mean refractive indices and the film thickness. On the basis of thus-calculated nx, ny and nz, Nz=(nx−nz)/(nx−ny) is further calculated.

In this specification, the wavelength at which the refraction index is measured is 550 nm unless otherwise specified.

### EXAMPLES

Paragraphs below will further specifically describe features of the present invention, referring to Examples and Comparative Examples. Any materials, amount of use, ratio, details of processing, procedures of processing and so forth shown in Examples may appropriately be modified without departing from the spirit of the present invention. Therefore, it is to be understood that the scope of the present invention should not be interpreted in a limited manner based on the specific examples shown below.

### Synthesis of Polymerizable Liquid Crystal Compound of this Invention

### Example 1

Compound (1) was synthesized according to the scheme below:

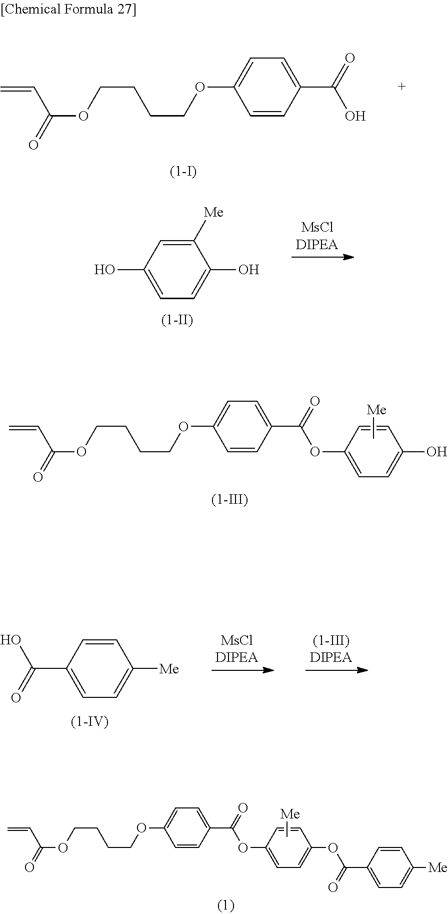

BHT (37 mg) was added to a tetrahydrofuran (THF) solution (20 mL) containing methanesulfonyl chloride (10.22 g), and the inner temperature was cooled down to −5° C. To the mixture, a THF solution (50 mL) containing 1-I (31.5 mmol, 8.33 g) and diisopropylethylamine (17.6 mL) was added dropwise, so as not to elevate the inner temperature to 0° C. or above. The mixture was stirred at −5° C. for 30 minutes, and thereto diisopropylethylamine (16.7 mL) and a THF solution (20 mL) containing and 1-II, and 4-dimethylaminopyridine (DMAP) (one spatula) were added. The mixture was then stirred at room temperature for 4 hours. To the mixture added was methanol (5 mL) to terminate the reaction, and further added were water and ethyl acetate. An organic layer as a result of extraction with ethyl acetate was evaporated using a rotary evaporator to remove the solvent, and the residue was purified by silica gel column chromatography, to obtain 1-III.

BHT (3 mg) was added to a THF solution (10 mL) containing methanesulfonyl chloride (355 mg), and the inner temperature was cooled down to −5° C. To the mixture, carboxylic acid 1-IV (404 mg) and diisopropylethylamine (472 μL) were added dropwise, so as not to elevate the inner temperature to 0° C. or above. The mixture was stirred at −5° C. for 30 minutes, and thereto diisopropylethylamine (472 μL) and a THF solution (2 mL) containing phenol 1-III (1.0 g), and DMAP (one spatula) were added. The mixture was then stirred at room temperature for two hours. Methanol (5 mL) was then added to the mixture to terminate the reaction, followed by further addition of water and ethyl acetate. An organic layer as a result of extraction with ethyl acetate was evaporated using a rotary evaporator to remove the solvent, to obtain a crude product of compound (1). Purification by silica gel column chromatography gave compound (1) in a yield of 58%. ¹H-NMR (solvent: CDCl₃) δ (ppm): 1.9-2.0 (m, 4H), 2.2 (s, 3H), 2.5 (s, 3H), 4.1-4.3 (m, 4H), 5.8 (d, 1H), 6.1 (dd, 1H), 6.4 (d, 1H), 6.9-7.0 (m, 2H), 7.1-7.2 (m, 3H), 7.3-7.4 (m, 2H), 8.1-8.2 (m, 4H)

Phase transition temperatures of the compound (1) were determined by texture observation under a polarizing microscope. Transition from crystal phase to nematic liquid crystal phase was observed at 83° C., and transition into isotropic liquid phase was observed above 135° C.

### Example 2

Compound (2) was obtained according to the synthetic method same as in Example 1, except that p-ethylbenzoic acid was used. Also compound (2) showed the nematic liquid crystallinity same as compound (1).

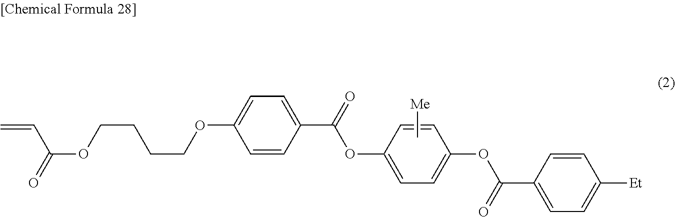

¹H-NMR (solvent: CDCl₃) δ (ppm): 1.3 (t, 3H), 1.9-2.0 (m, 4H), 2.3 (s, 3H), 2.7-2.8 (m, 2H), 4.1-4.3 (m, 4H), 5.8 (d, 1H), 6.1 (dd, 1H), 6.4 (d, 1H), 6.9-7.0 (m, 2H), 7.1-7.2 (m, 3H), 7.3-7.4 (m, 2H), 8.1-8.2 (m, 4H)

### Example 3

Compound (3) was obtained according to the synthetic method same as in Example 1, except that p-n-propylbenzoic acid was used. Also compound (3) showed the nematic liquid crystallinity same as compound (1).

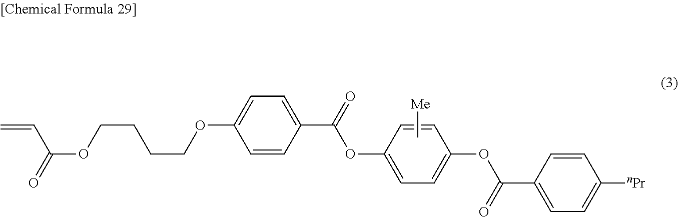

¹H-NMR (solvent: CDCl₃) δ (ppm): 1.0 (t, 3H), 1.6-1.8 (m, 2H), 1.9-2.0 (m, 4H), 2.3 (s, 3H), 2.7-2.8 (m, 2H), 4.1-4.3 (m, 4H), 5.8 (d, 1H), 6.1 (dd, 1H), 6.4 (d, 1H), 6.9-7.0 (m, 2H), 7.1-7.2 (m, 3H), 7.3-7.4 (m, 2H), 8.1-8.2 (m, 4H)

### Example 4

Compound (4) was obtained according to the synthetic method same as in Example 1, except that p-n-butylbenzoic acid was used. Also compound (4) showed the nematic liquid crystallinity same as compound (1).

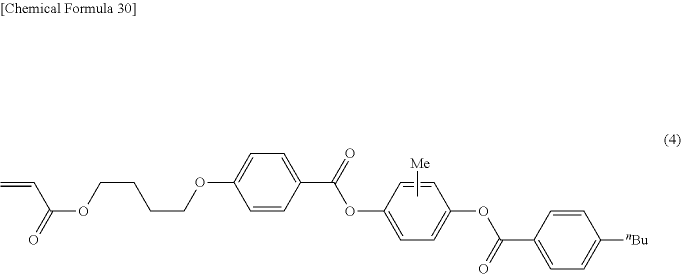

¹H-NMR (solvent: CDCl₃) δ (ppm): 0.9 (t, 3H), 1.3-1.5 (m, 2H), 1.6-1.7 (m, 2H), 1.9-2.0 (m, 4H), 2.3 (s, 3H), 2.7-2.8 (m, 2H), 4.1-4.3 (m, 4H), 5.8 (d, 1H), 6.1 (dd, 1H), 6.4 (d, 1H), 6.9-7.0 (m, 2H), 7.1-7.2 (m, 3H), 7.3-7.4 (m, 2H), 8.1-8.2 (m, 4H)

### Example 5

Compound (5) was obtained according to the synthetic method same as in Example 1, except that p-methoxybenzoic acid was used. Also compound (5) showed the nematic liquid crystallinity same as compound (1).

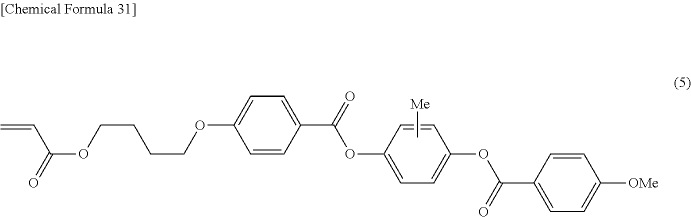

¹H-NMR (solvent: CDCl₃) δ (ppm): 1.9-2.0 (m, 4H), 2.2 (s, 3H), 3.9 (s, 3H), 4.1-4.3 (m, 4H), 5.8 (d, 1H), 6.1 (dd, 1H), 6.4 (d, 1H), 6.9-7.0 (m, 4H), 7.1-7.2 (m, 3H), 8.1-8.2 (m, 4H)

### Example 6

Compound (6) was obtained according to the synthetic method same as in Example 1, except that p-ethoxybenzoic acid was used. Also compound (6) showed the nematic liquid crystallinity same as compound (1).

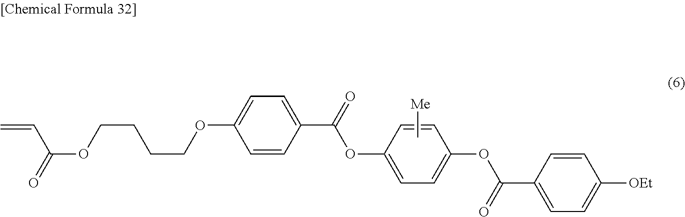

¹H-NMR (solvent: CDCl₃) δ (ppm): 1.5 (t, 3H), 1.9-2.0 (m, 4H), 2.3 (s, 3H), 4.0-4.3 (m, 6H), 5.8 (d, 1H), 6.1 (dd, 1H), 6.4 (d, 1H), 6.9-7.0 (m, 4H), 7.1-7.2 (m, 3H), 8.1-8.2 (m, 4H)

### Example 7

Compound (7) was obtained according to the synthetic method same as in Example 1, except that p-phenylbenzoic acid was used. Also compound (7) showed the nematic liquid crystallinity same as compound (1).

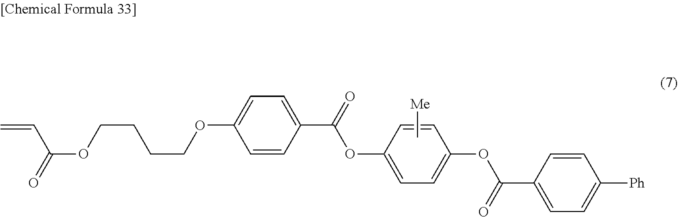

¹H-NMR (solvent: CDCl₃) δ (ppm): 1.9-2.0 (m, 4H), 2.3 (s, 3H), 4.1-4.3 (m, 4H), 5.8 (d, 1H), 6.1 (dd, 1H), 6.4 (d, 1H), 6.9-7.0 (m, 2H), 7.1-7.3 (m, 3H), 7.4-7.5 (m, 3H), 7.6-7.8 (m, 4H), 8.1-8.3 (m, 4H)

### Example 8

Compound (8) was obtained according to the synthetic method same as in Example 1, except that p-methoxycinnamic acid was used. Also compound (8) showed the nematic liquid crystallinity same as compound (1).

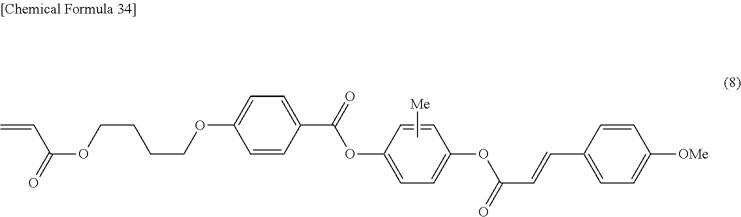

¹H-NMR (solvent: CDCl₃) δ (ppm): 1.9-2.0 (m, 4H), 2.2 (s, 3H), 3.9 (s, 3H), 4.1-4.3 (m, 4H), 5.8 (d, 1H), 6.1 (dd, 1H), 6.4-6.6 (m, 2H), 6.9-7.0 (m, 4H), 7.1-7.2 (m, 3H), 7.5-7.6 (m, 2H), 7.8-7.9 (m, 1H), 8.1-8.2 (m, 2H)

### Example 9

Compound (9) was obtained according to the synthetic method same as in Example 1, except that cinnamic acid was used. Also compound (9) showed the nematic liquid crystallinity same as compound (1).

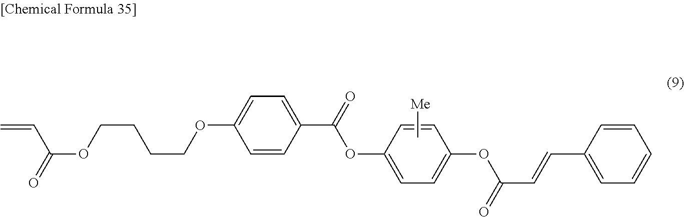

¹H-NMR (solvent: CDCl₃) δ (ppm): 1.9-2.0 (m, 4H), 2.2 (s, 3H), 4.1-4.3 (m, 4H), 5.8 (d, 1H), 6.1 (dd, 1H), 6.4 (d, 1H), 6.6-6.7 (d, 1H), 6.9-7.0 (m, 4H), 7.1-7.2 (m, 3H), 7.4-7.5 (m, 3H), 7.6-7.7 (m, 2H), 7.9 (d, 1H), 8.1-8.2 (m, 2H)

### Example 11

### Preparation of Liquid Crystal Composition of this Invention

Using compound (1) synthesized in Example 1, a liquid crystal composition was prepared according to the method below.

A coating liquid (A) of liquid crystal composition, having a composition below, was prepared, and used as a liquid crystal composition of Example 11.

| | |
|---|---|
| Compound (1) | 30 parts by mass |
| Bifunctional polymerizable liquid crystal | 70 parts by mass |
| compound (1-A), shown below | |
| MEK | 233 parts by mass |

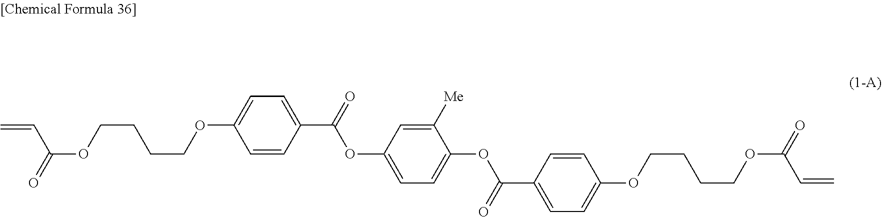 [SMILES: C=CC(=O)OCCCCOc1ccc(C(=O)Oc2ccc(OC(=O)c3ccc(OCCCCOC(=O)C=C)cc3)c(C)c2)cc1]

### <Manufacture of Film>

Next, a film of Example 11 was manufactured using the liquid crystal composition of Example 11.

On a cleaned glass substrate, polyimide alignment film SE-130 from Nissan Chemical Industries, Ltd. was formed by spin coating, dried, and baked at 250° C. for one hour. The obtained film was rubbed to thereby manufacture a substrate with alignment film. On the rubbed surface of the alignment film of the substrate, coating liquid (A) of liquid crystalline composition which contains the liquid crystal composition of Example 11 was coated at room temperature by spin coating, and the coating was allowed to stand at room temperature for 30 minutes.

### (Evaluation of Suppressive Effect on Crystal Deposition)

An arbitrary region of the surface of liquid crystal film, in the film of Example 11, was visually observed under a polarizing microscope, to find a ratio of crystal deposition of 10%.

### Examples 12 to 19 and Comparative Examples 21 to 26

Coating liquids of liquid crystal compositions were prepared in the same way as in Example 1, except that the compounds listed in Table 1 below were used in place of compound (1) manufactured in Example 1, which were denoted as liquid crystal compositions of the individual Examples and Comparative Examples.

Films of the individual Examples and Comparative Examples were manufactured in the same way as in Example 11, except that the liquid crystal compositions of the individual Examples and Comparative Examples were used in place of the liquid crystal composition of Example 11.

The ratio of crystal deposition of the obtained films of the individual Examples and Comparative Examples was measured. Results were as summarized in Table 1.

TABLE 1

| | monofunctional | Bifunctional | crystal |
|---|---|---|---|
| | polymerizable | polymerizable | deposition |
| | liquid crystal | liquid crystal | properties of |
| | compound | compound | film |
| | 30 parts by mass | 70 parts by mass | (30:70 mixing) |
| Example 11 | compound (1) | polymerizable liquid | 3 |
| | | crystal compound | |
| | | (1-A) | |
| Example 12 | compound (2) | polymerizable liquid | 3 |
| | | crystal compound | |
| | | (1-A) | |
| Example 13 | compound (3) | polymerizable liquid | 3 |
| | | crystal compound | |
| | | (1-A) | |
| Example 14 | compound (4) | polymerizable liquid | 3 |
| | | crystal compound | |
| | | (1-A) | |
| Example 15 | compound (5) | polymerizable liquid | 3 |
| | | crystal compound | |
| | | (1-A) | |
| Example 16 | compound (6) | polymerizable liquid | 2 |
| | | crystal compound | |
| | | (1-A) | |
| Example 17 | compound (7) | polymerizable liquid | 3 |
| | | crystal compound | |
| | | (1-A) | |
| Example 18 | compound (8) | polymerizable liquid | 3 |
| | | crystal compound | |
| | | (1-A) | |
| Example 19 | compound (9) | polymerizable liquid | 3 |
| | | crystal compound | |
| | | (1-A) | |
| Comparative | comparative | polymerizable liquid | 1 |
| Example 21 | example | crystal compound | |
| | compound (1′) | (1-A) | |
| Comparative | comparative | polymerizable liquid | 1 |
| Example 22 | example | crystal compound | |
| | compound (2′) | (1-A) | |
| Comparative | comparative | polymerizable liquid | 1 |
| Example 23 | example | crystal compound | |
| | compound (3′) | (1-A) | |
| Comparative | comparative | polymerizable liquid | 1 |
| Example 24 | example | crystal compound | |
| | compound (4′) | (1-A) | |
| Comparative | comparative | polymerizable liquid | 1 |
| Example 25 | example | crystal compound | |
| | compound (5′) | (1-A) | |
| Comparative | comparative | polymerizable liquid | 1 |
| Example 26 | example | crystal compound | |
| | compound (6′) | (1-A) | |

In Table 1 above, crystal deposition properties was assigned with “3” if the area of crystal deposition visually accounts for 0 to 20% of the film, assigned with “2” if exceeds 20% and falls under 50%, and assigned with “1” if exceeds 50%.

Structures of Comparative Example Compounds (1′) to (6′) in Table 1 are listed below. Note that Comparative Example Compound (2′) is a compound described in Japanese Translation of PCT International Application Publication No. JP-T2-2002-536529, and Comparative Example Compound (3′) is a compound described in Molecular Crystals and Liquid Crystals (2010), 530 169-174.

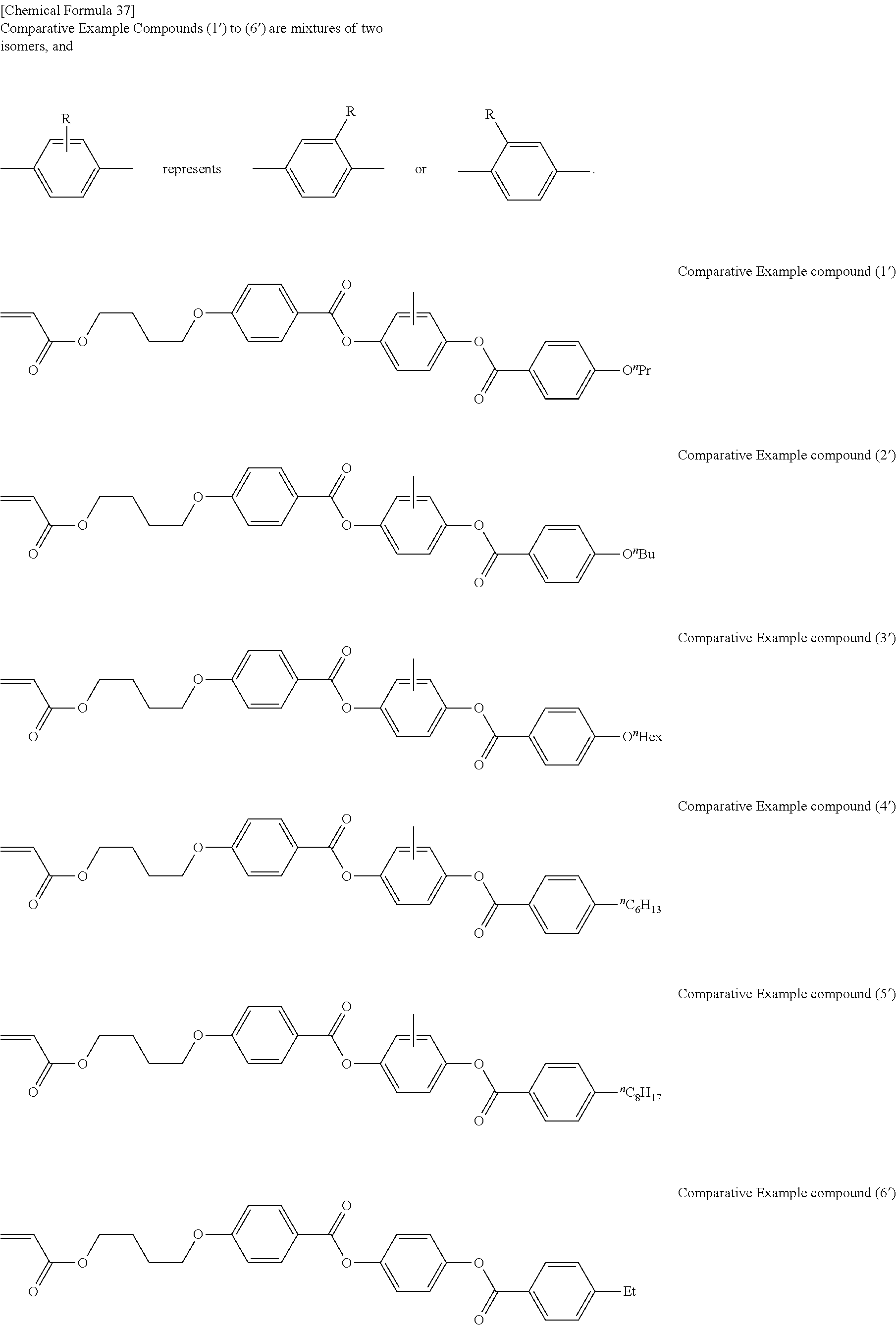

Results of Examples 11 to 19 and Comparative Examples 21 to 26 summarized in Table 1 showed that addition of the polymerizable liquid crystal compounds represented by the formula (1) of this invention, synthesized in Examples 1 to 9, sharply reduced the crystal deposition of the polymerizable liquid crystal compound (1-A), as compared with addition of the conventional monofunctional polymerizable liquid crystal compound which is not covered by the formula (1).

### Examples 31 to 39 and Comparative Examples 41 to 45

Liquid crystal compositions were prepared in the same way as in Example 11, except that the liquid crystal composition in Example 11 was altered to the liquid crystal compositions having the compositional ratio below, which were denoted as the liquid crystal compositions of the individual Examples and Comparative Examples.

Films of the individual Examples and Comparative Examples were manufactured in the same way as in Example 11, except that the liquid crystal compositions of the individual Examples and Comparative Examples were used in place of the liquid crystal composition of Example 11.

The obtained films of the individual Examples and Comparative Examples were measured in terms of the ratio of crystal deposition. Results were as summarized in Table 2 below.

| | |
|---|---|
| Compounds (1) to (9) | 20 parts by mass |
| Polymerizable liquid crystal compound (1-A), | 80 parts by mass |
| described above | |
| MEK | 233 parts by mass |

TABLE 2

| | monofunctional | | |
|---|---|---|---|
| | polymerizable | Bifunctional | crystal |
| | liquid crystal | polymerizable | deposition |
| | compound | liquid crystal | properties of |
| | 20 parts | compound | film |
| | by mass | 80 parts by mass | (20:80 mixing) |
| Example 31 | compound (1) | polymerizable liquid | 2 |
| | | crystal compound (1-A) | |
| Example 32 | compound (2) | polymerizable liquid | 3 |
| | | crystal compound (1-A) | |
| Example 33 | compound (3) | polymerizable liquid | 2 |
| | | crystal compound (1-A) | |
| Example 34 | compound (4) | polymerizable liquid | 2 |
| | | crystal compound (1-A) | |
| Example 35 | compound (5) | polymerizable liquid | 2 |
| | | crystal compound (1-A) | |
| Example 36 | compound (6) | polymerizable liquid | 2 |
| | | crystal compound (1-A) | |
| Example 37 | compound (7) | polymerizable liquid | 3 |
| | | crystal compound (1-A) | |
| Example 38 | compound (8) | polymerizable liquid | 2 |
| | | crystal compound (1-A) | |
| Example 39 | compound (9) | polymerizable liquid | 2 |
| | | crystal compound (1-A) | |
| Comparative | comparative | polymerizable liquid | 1 |
| Example 41 | compound (1′) | crystal compound (1-A) | |
| Comparative | comparative | polymerizable liquid | 1 |
| Example 42 | compound (2′) | crystal compound (1-A) | |
| Comparative | comparative | polymerizable liquid | 1 |
| Example 43 | compound (3′) | crystal compound (1-A) | |
| Comparative | comparative | polymerizable liquid | 1 |
| Example 44 | compound (4′) | crystal compound (1-A) | |
| Comparative | comparative | polymerizable liquid | 1 |
| Example 45 | compound (5′) | crystal compound (1-A) | |

In Table 2 above, crystal deposition properties was assigned with “3” if the area of crystal deposition visually accounts for 0 to 20% of the film, assigned with “2” if exceeds 20% and falls under 50%, and assigned with “1” if exceeds 50%.

From the results of Examples 31 to 39 summarized in Table 2, it was found that, among the polymerizable liquid crystal compounds (1) to (9) of this invention represented by the formula (1), in particular compounds (2) and (7) demonstrated strong suppressive effects on crystal deposition. While not adhering to any theory, a strong suppressive effect on crystal deposition shown by compound (7) is ascribable to that the crystal form achieved when the liquid crystal composition crystallizes is difficult to crystallize.

### Example 78

Compound (1K) was obtained according to the synthetic method same as in Example 1, except that 4-(acryloylamino)benzoic acid was used.

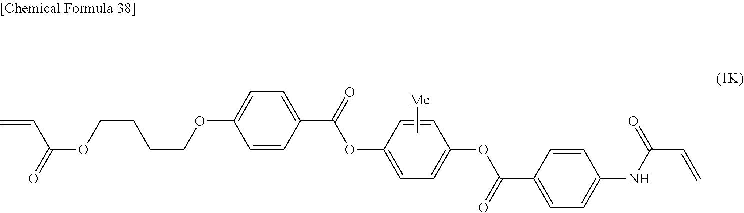

¹H-NMR (solvent: CDCl₃) δ (ppm): 1.9-2.0 (m, 4H), 2.25 (s, 3H), 4.1-4.3 (m, 4H), 5.8-5.9 (m, 2H), 6.1-6.2 (m, 1H), 6.3-6.5 (m, 3H), 6.9-7.0 (m, 2H), 7.1-7.2 (m, 3H), 7.6-7.7 (m, 2H), 7.8 (s, 1H), 8.1-8.2 (m, 4H)

### Example 79

Compound (2K) was obtained according to the synthetic method same as in Example 1, except that 4-(methacryloylamino)benzoic acid was used.

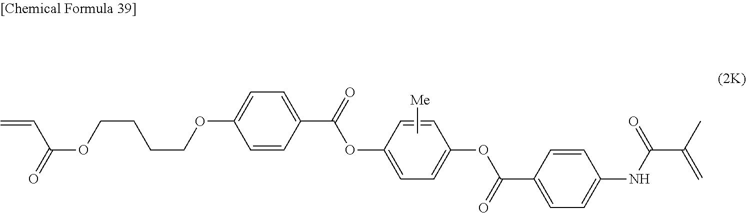

¹H-NMR (solvent: CDCl₃) δ (ppm): 1.9-2.0 (m, 4H), 2.05 (s, 3H) 2.25 (s, 3H), 4.1-4.3 (m, 4H), 5.5 (s, 1H), 5.8-5.9 (m, 2H), 6.1 (dd, 1H), 6.4 (d, 1H), 6.9-7.0 (m, 2H), 7.1-7.2 (m, 3H), 7.7-7.8 (m, 2H) 8.0 (s, 1H), 8.1-8.2 (m, 4H)

### Example 80

Compound (3K) was obtained according to the synthetic method same as in Example 1, except that 4-(allyloxy carbamoyl)benzoic acid was used.

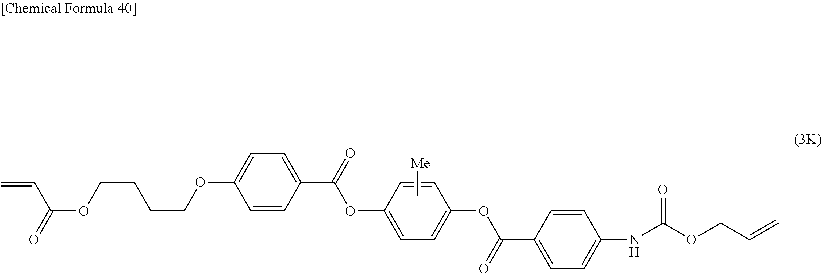

H-NMR (solvent: CDCl₃) δ (ppm): 1.9-2.0 (m, 4H), 2.25 (s, 3H), 4.1-4.3 (m, 4H), 4.7 (m, 2H), 5.25-5.45 (m, 2H), 5.8 (d, 1H), 5.9-6.0 (m, 1H), 6.15 (dd, 1H), 6.4 (d, 1H), 6.9-7.0 (m, 2H), 7.1-7.2 (m, 3H), 7.4 (m, 1H), 7.45-7.55 (m, 2H), 8.1-8.2 (m, 4H)

### Example 81

Compound (6K) was obtained according to the synthetic method same as in Example 1, except that 4-allyloxy benzoic acid was used.

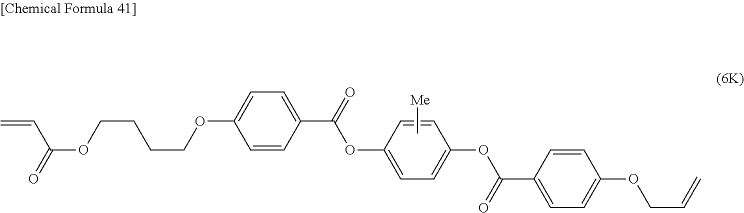

H-NMR (solvent: CDCl₃) δ (ppm): 1.9-2.0 (m, 4H), 2.25 (s, 3H), 4.1-4.3 (m, 4H), 4.65 (m, 2H), 5.3-5.5 (m, 2H), 5.8 (d, 1H), 6.0-6.1 (m, 1H), 6.15 (dd, 1H), 6.4 (d, 1H), 6.9-7.0 (m, 4H), 7.1-7.2 (m, 3H), 8.1-8.2 (m, 4H)

### Example 82

Compound (7K) was obtained according to the synthetic method same as in Example 1, except that 4-vinyl benzoic acid was used.

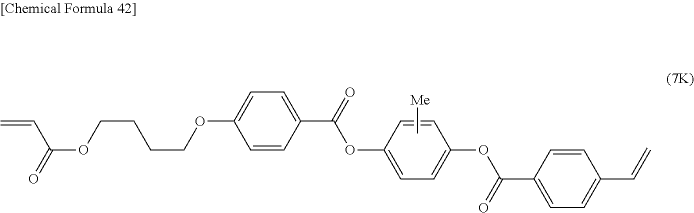

H-NMR (solvent: CDCl₃) δ (ppm): 1.9-2.0 (m, 4H), 2.25 (s, 3H), 4.1-4.3 (m, 4H), 5.5 (d, 1H), 5.8-5.9 (d, 1H), 5.95 (d, 1H), 6.15 (dd, 1H), 6.4 (d, 1H), 6.75-6.9 (m, 1H), 6.9-7.0 (m, 2H), 7.1-7.2 (m, 3H), 7.5-7.6 (m, 2H), 8.1-8.2 (m, 4H)

### Example 83

Compound (8K) was obtained according to the synthetic method same as in Example 1, except that 4-[N-(2-methacryloyloxyethyl)carbamoyloxy]benzoic acid was used.

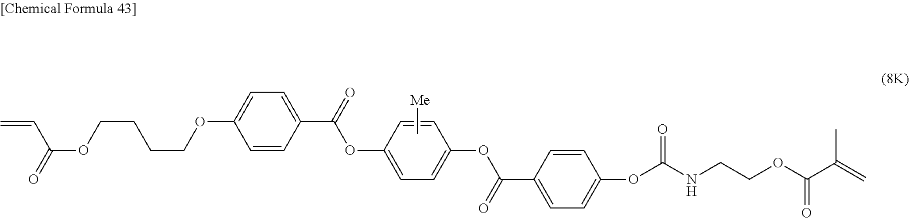

H-NMR (solvent: CDCl₃) δ (ppm): 1.9-2.0 (m, 4H), 2.0 (s, 3H), 2.25 (s, 3H), 3.6-3.7 (m, 2H), 4.1-4.4 (m, 6H), 5.4 (bd, 1H), 5.65 (d, 1H), 5.8-5.9 (d, 2H), 6.15 (dd, 1H), 6.4 (d, 1H), 6.9-7.0 (m, 4H), 7.1-7.2 (m, 3H), 8.1-8.2 (m, 4H)

### Example 84

Compound (9K) was obtained according to the synthetic method same as in Example 1, except that 4-[N-(2-acryloyloxyethyl)carbamoyloxy]benzoic acid was used.

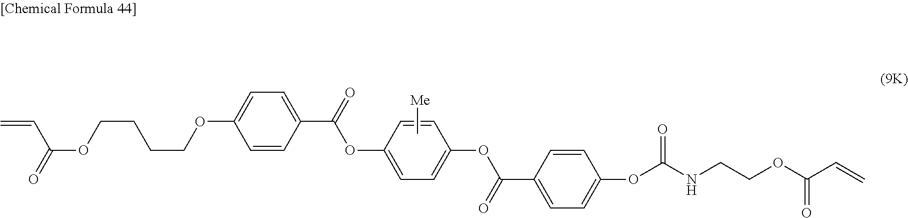

H-NMR (solvent: CDCl₃) δ (ppm): 1.9-2.0 (m, 4H), 2.25 (s, 3H), 3.6-3.7 (m, 2H), 4.1-4.4 (m, 6H), 5.8-5.9 (m, 2H), 6.1-6.2 (m, 2H), 6.3-6.5 (m, 2H), 6.9-7.0 (m, 4H), 7.1-7.2 (m, 3H), 8.1-8.2 (m, 4H)

### Example 85

Compound (4K) was obtained according to the synthetic method same as in Example 1, except that 4-(ethyl yloxycarbamoyl)benzoic acid was used.

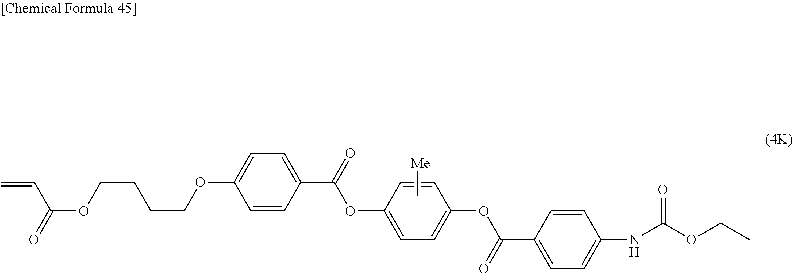

H-NMR (solvent: CDCl₃) δ (ppm): 1.3 (t, 3H), 1.9-2.0 (m, 4H), 2.25 (s, 3H), 4.1-4.3 (m, 6H), 5.8 (d, 1H), 6.15 (dd, 1H), 6.4 (d, 1H), 6.9-7.0 (m, 2H), 7.1-7.2 (m, 3H), 7.45-7.55 (m, 2H), 8.1-8.2 (m, 4H)

### Examples 86 to 93

Liquid crystal compositions were prepared in the same way as in Example 11, except that the liquid crystal composition in Example 11 was altered to the liquid crystal compositions containing the compounds (1K) to (4K), and (6K) to (9K) according to the compositional ratio below, which were denoted as liquid crystal compositions of the individual Examples.

Films of the individual Examples were manufactured in the same way as in Example 11, except that the liquid crystal compositions of the individual Examples and Comparative Examples were used in place of the liquid crystal composition of Example 11.

The obtained films of the individual Examples were measured in terms of ratio of crystal deposition. Results were as summarized in Table 3 below.

TABLE 3

| | monofunctional | | |
|---|---|---|---|
| | polymerizable | | crystal |
| | liquid crystal | Bifunctional | deposition |
| | compound | polymerizable | properties of |
| | 20 parts | liquid crystal compound | film |
| | by mass | 80 parts by mass | (20:80 mixing) |
| Example 86 | compound (1K) | polymerizable liquid | 3 |
| | | crystal compound (1-A) | |
| Example 87 | compound (2K) | polymerizable liquid | 3 |
| | | crystal compound (1-A) | |
| Example 88 | compound (3K) | polymerizable liquid | 2 |
| | | crystal compound (1-A) | |
| Example 89 | compound (6K) | polymerizable liquid | 3 |
| | | crystal compound (1-A) | |
| Example 90 | compound (7K) | polymerizable liquid | 2 |
| | | crystal compound (1-A) | |
| Example 91 | compound (8K) | polymerizable liquid | 2 |
| | | crystal compound (1-A) | |
| Example 92 | compound (9K) | polymerizable liquid | 2 |
| | | crystal compound (1-A) | |
| Example 93 | compound (4K) | polymerizable liquid | 2 |
| | | crystal compound (1-A) | |

In Table 3 above, crystal deposition properties was assigned with “3” if the area of crystal deposition visually accounts for 0 to 20% of the film, assigned with “2” if exceeds 20% and falls under 50%, and assigned with “1” if exceeds 50%.

From the results summarized in Table 3, it was found that, among the polymerizable liquid crystal compounds of this invention represented by the formula (1), in particular compounds (1K), (2K) and (6K) showed distinctive suppressive effect on crystal deposition.

### Example 51

Liquid crystal composition (B) was prepared using compound (1), according to the method below:

| | |
|---|---|
| Compound (1) | 20 parts by mass |
| Polymerizable liquid crystal compound (1-A) | 80 parts by mass |
| Paliocolor LC756 (from BASF), chiral agent | 3 parts by mass |
| Air interface aligning agent (X1-1) | 0.04 parts by mass |
| IRGACURE 819(from BASF), polymerization | 3 parts by mass |
| initiator | |
| Chloroform, solvent | 300 parts by mass |

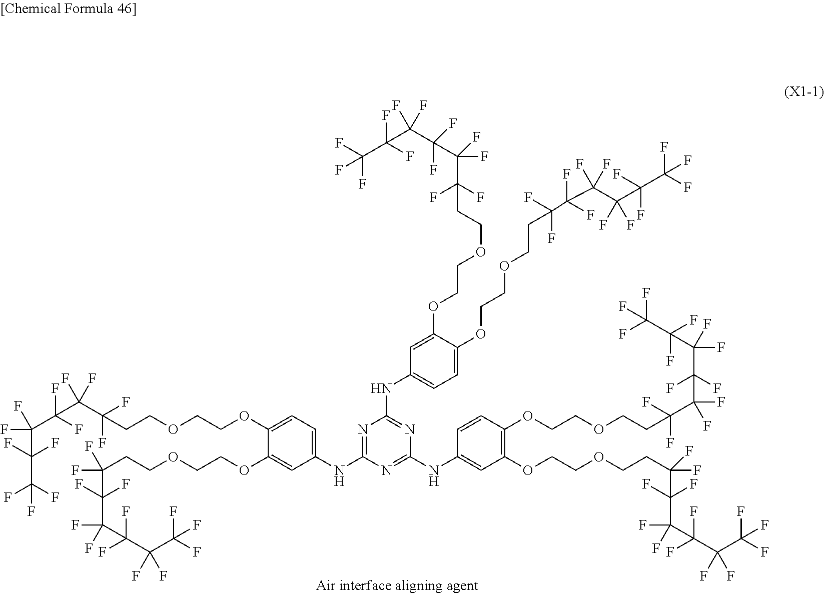 [SMILES: FC(F)(F)C(F)(F)C(F)(F)C(F)(F)C(F)(F)C(F)(F)CCOCCOc1ccc(Nc2nc(Nc3ccc(OCCOCCC(F)(F)C(F)(F)C(F)(F)C(F)(F)C(F)(F)C(F)(F)F)c(OCCOCCC(F)(F)C(F)(F)C(F)(F)C(F)(F)C(F)(F)C(F)(F)F)c3)nc(Nc3ccc(OCCOCCC(F)(F)C(F)(F)C(F)(F)C(F)(F)C(F)(F)C(F)(F)F)c(OCCOCCC(F)(F)C(F)(F)C(F)(F)C(F)(F)C(F)(F)C(F)(F)F)c3)n2)cc1OCCOCCC(F)(F)C(F)(F)C(F)(F)C(F)(F)C(F)(F)C(F)(F)F]

On the surface of the alignment film of the substrate with alignment film, manufactured in the same way as in Example 11, liquid crystal composition (B) was coated at room temperature by spin coating, the coating was aged at 120° C. for 3 minutes, irradiated with light at room temperature using a high-pressure mercury lamp, with short wavelength UV components cut off, for 10 seconds to fix the alignment, to thereby obtain a selective reflection film. No crystal deposition in the coated film was observed over the period after the coating and before the heating.

The obtained selective reflection film was observed under a polarizing microscope, and was found to show uniform alignment without alignment defect. Further analysis of transmission spectrum using a spectrophotometer UV-3100PC from Shimadzu Corporation showed a selective reflection peak in the infrared region.

### Examples 52 to 59

Coating liquids of liquid crystalline compositions were respectively prepared in the same way as in Example 51, except that compound (2) to compound (9) were used in place of compound (1). These coating liquids were used to respectively form selective reflection films in the same way as in Example 51. All of these selective reflection films showed good alignability. Measurement of transmission spectra using a spectrophotometer UV-3100PC showed selective reflection peaks in the infrared region.

### Examples 94 to 98

Coating liquids of liquid crystalline compositions were respectively prepared in the same way as in Example 51, except that compounds (1K) to (3K), (6K) and (8K) were used in place of compound (1). These coating liquids were used to respectively form selective reflection films in the same way as in Example 51. All of these selective reflection films showed good alignability. Measurement of transmission spectra using the spectrophotometer UV-3100PC showed selective reflection peaks in the infrared region.

### Example 60

### Fabrication of Optical Compensation Film (1)

A coating liquid of liquid crystalline composition (C) was prepared using compound (1) according to the method below:

| | |
|---|---|
| Compound (1) | 20 parts by mass |
| Polymerizable compound (1-A) | 80 parts by mass |
| IRGACURE 819 (from BASF), polymerization | 3 parts by mass |
| initiator | |
| Air interface aligning agent (X1-2) | 0.1 parts by mass |
| Methyl ethyl ketone, solvent | 400 parts by mass |

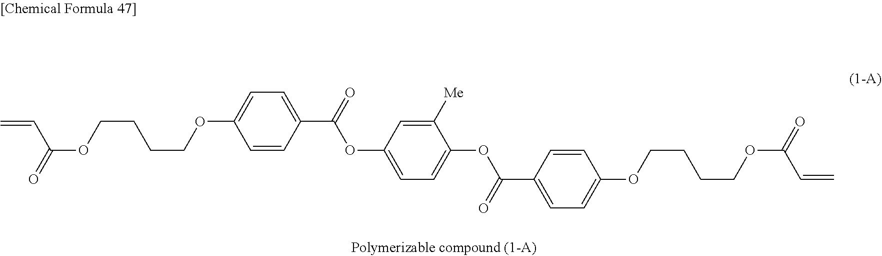 [SMILES: C=CC(=O)OCCCCOc1ccc(C(=O)Oc2ccc(OC(=O)c3ccc(OCCCCOC(=O)C=C)cc3)c(C)c2)cc1]

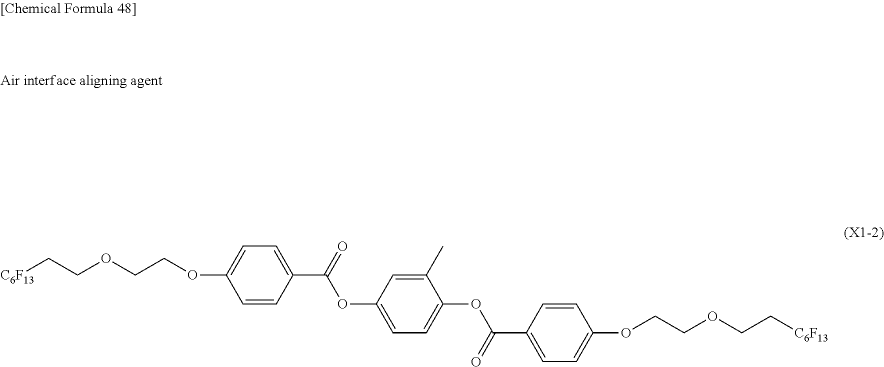

On a cleaned glass substrate, polyimide alignment film SE-130 from Nissan Chemical Industries, Ltd. was formed by spin coating, dried, and baked at 250° C. for one hour. The obtained film was rubbed to thereby manufacture a substrate with alignment film. On the surface of the substrate, coating liquid (C) of liquid crystalline composition was coated at room temperature by spin coating, the coating was aged for alignment at 60° C. for one minute, and then irradiated with light at room temperature using a high-pressure mercury lamp, with short wavelength UV components cut off, for 10 seconds to fix the alignment, to thereby form an optically anisotropic layer. No crystal deposition in the coated film was observed over the period after the coating and before the heating.

The obtained optical compensation film was observed under a polarizing microscope, and was found to show uniform alignment without alignment defect. Further analysis of transmission spectrum using a spectrophotometer UV-3100PC from Shimadzu Corporation showed a selective reflection peak in the infrared region.

Further measurement of retardation (Re) of the obtained optical compensation film, using AxoScan (Mueller matrix polarimeter) from Axometrics, Inc., showed an Re at 550 nm of 162.4 nm.

### Examples 61 to 68

Coating liquids of liquid crystalline compositions were respectively prepared in the same way as in Example 60, except that compound (2) to compound (9) were used in place of compound (1). Optical compensation films were formed by respectively using these coating liquids, in the same way as in Example 60. The obtained optical compensation films were observed under a polarizing microscope, and were found to show uniform alignment without alignment defect. Further measurement of retardation (Re) at 550 nm and thickness of the obtained optical compensation films, were as summarized below.

TABLE 4

| | | | Thickness |
|---|---|---|---|
| | compound | Re (nm) | (μm) |
| Example 60 | compound (1) | 162.4 | 1.02 |
| Example 61 | compound (2) | 161.3 | 1.01 |
| Example 62 | compound (3) | 164.4 | 1.01 |
| Example 63 | compound (4) | 163.6 | 1.03 |
| Example 64 | compound (5) | 166.2 | 1.00 |
| Example 65 | compound (6) | 166.8 | 1.01 |
| Example 66 | compound (7) | 183.2 | 1.03 |
| Example 67 | compound (8) | 193.6 | 1.02 |
| Example 68 | compound (9) | 160.3 | 1.02 |

### Examples 99 to 103

Coating liquids of liquid crystalline compositions were respectively prepared in the same way as in Example 60, except that compounds (1K) to (3K), and (6K) to (8K) were used in place of compound (1). Optical compensation films were formed by respectively using these coating liquids, in the same way as in Example 60. The obtained optical compensation films were observed under a polarizing microscope, and were found to show uniform alignment without alignment defect. Further measurement of retardation (Re) at 550 nm and thickness of the obtained optical compensation films, were as summarized below.

TABLE 5

| | | | Thickness |
|---|---|---|---|
| | compound | Re (nm) | (μm) |
| Example 99 | compound (1K) | 180.4 | 1.00 |
| Example 100 | compound (2K) | 167.3 | 1.01 |
| Example 101 | compound (3K) | 170.8 | 0.99 |
| Example 102 | compound (6K) | 162.1 | 1.01 |
| Example 103 | compound (8K) | 130.1 | 1.00 |

### Example 69

### Fabrication of Optical Compensation Film (2)

Coating liquid of liquid crystalline composition (D) was prepared using compound (1), according to the method described below:

| | |
|---|---|
| Compound (1) | 20 parts by mass |
| Polymerizable compound (1-A) | 80 parts by mass |
| Sensitizer (Kayacure DETX, from Nippon | 1 part by mass |
| Kayaku Co., Ltd.) | |
| Air interface aligning agent (X1-3) | 0.11 parts by mass |
| Onium salt (X1-4) | 1.5 parts by mass |
| Methyl ethyl ketone, solvent | 300 parts by mass |

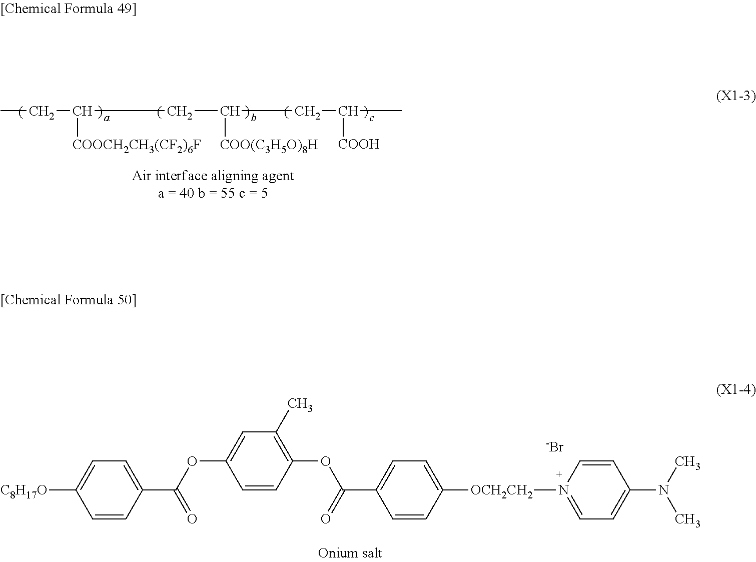

| | |
|---|---|
| Composition of Coating Liquid for Forming Alignment | 10 parts by mass |
| Film Modified polyvinyl alcohol, shown below | |
| Water | 371 parts by mass |
| Methanol | 119 parts by mass |
| Glutaraldehyde | 0.5 parts by mass |

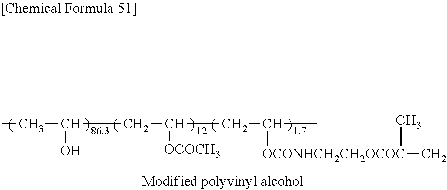

On a cleaned glass substrate, the coating liquid for forming alignment film was coated using a wire bar coater in an amount of 20 mL/m². The coating was dried under hot air at 60° C. for 60 seconds, and further under hot air at 100° C. for 120 seconds, to thereby fabricate a substrate with alignment film. Over the surface of the substrate, coating liquid of liquid crystalline composition (D) was coated at room temperature by spin coating, the coating was aged for alignment at 60° C. for one minute, and then irradiated with light at 50° C. using a high-pressure mercury lamp, with short wavelength UV components cut off, for 10 seconds to fix the alignment, to thereby form an optical compensation film. No crystal deposition in the coated film was observed over the period after the coating and before the heating.

The obtained optical compensation film was observed under a polarizing microscope, and was found to show uniform alignment without alignment defect.

Further measurement of Rth of the obtained optical compensation film, using AxoScan (Mueller matrix polarimeter) from Axometrics, Inc., showed an Rth at 550 nm of −124.8 nm.

### Examples 70 to 77

Coating liquids of liquid crystalline compositions were respectively prepared in the same way as in Example 69, except that compound (2) to compound (9) were used in place of compound (1). Optical compensation films were formed by respectively using these coating liquids, in the same way as in Example 69. The obtained optical compensation films were observed under a polarizing microscope, and were found to show uniform alignment without alignment defect. Further measurement of Rth at 550 nm and thickness of the obtained optical compensation films, were as summarized below.

TABLE 6

| | compound | Rth (nm) | Thickness (μm) |
|---|---|---|---|
| Example 69 | compound (1) | −124.8 | 1.51 |
| Example 70 | compound (2) | −123.4 | 1.49 |
| Example 71 | compound (3) | −126.8 | 1.49 |
| Example 72 | compound (4) | −125.3 | 1.48 |
| Example 73 | compound (5) | −128.2 | 1.50 |
| Example 74 | compound (6) | −128.3 | 1.47 |
| Example 75 | compound (7) | −140.9 | 1.51 |
| Example 76 | compound (8) | −145.6 | 1.52 |
| Example 77 | compound (9) | −120.6 | 1.48 |

### Examples 104 to 108

Coating liquids of liquid crystalline compositions were respectively prepared in the same way as in Example 69, except that compounds (1K) to (3K), and (6K) to (8K) were used in place of compound (1). Optical compensation films were formed by respectively using these coating liquids, in the same way as in Example 69. The obtained optical compensation films were observed under a polarizing microscope, and were found to show uniform alignment without alignment defect. Further measurement of retardation (Re) at 550 nm and thickness of the obtained optical compensation films, were as summarized below.

TABLE 7

| | | | Thickness |
|---|---|---|---|
| | compound | Rth (nm) | (μm) |
| Example 104 | compound (1K) | −143.2 | 1.47 |
| Example 105 | compound (2K) | −135.1 | 1.47 |
| Example 106 | compound (3K) | −134.9 | 1.48 |
| Example 107 | compound (6K) | −127.5 | 1.46 |
| Example 108 | compound (7K) | −102.8 | 1.50 |

What is claimed is:

1. A polymerizable liquid crystal compound represented by the formula (1) below: 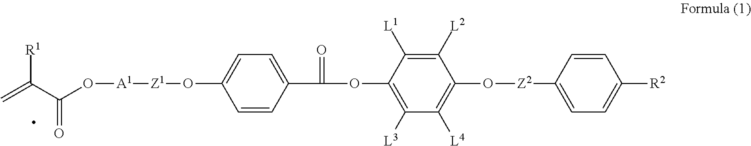
wherein A¹ represents a C₂₋₁₈ methylene group, one CH₂ or two or more non-adjacent (CH₂)s in the methylene group may be substituted by —O—;
Z¹ represents —CO—, —O—CO— or a single bond;
Z² represents —CO— or —CO—CH═CH—;
R¹ represents a hydrogen atom or methyl group;
R² represents hydrogen, C₁₋₄ straight-chain alkyl group, C₁ or C₂ straight-chain alkoxy group, phenyl group, aryloxy group, vinyl group, acryloylamino group, methacryloylamino group, N-aryloxycarbamoyl group, N-alkyloxycarbamoyl group having a C₁₋₄ alkyl group, N-(2-methacryloyloxyethyl)carbamoyloxy group or N-(2-acryloyloxyethyl)carbamoyloxy group; and
each of L¹, L², L³ and L⁴ independently represents C₁₋₄ alkyl group, C₁₋₄ alkoxy group, C₂₋₅ alkoxycarbonyl group, C₂₋₄ acyl group, halogen atom or hydrogen atom, at least one of L¹, L², L³ and L⁴ represents a substituent other than hydrogen atom.

2. The polymerizable liquid crystal compound of claim 1, wherein in the formula (1), R² represents hydrogen atom, C₁₋₄ straight-chain alkyl group, C₁ or C₂ straight-chain alkoxy group or phenyl group.

3. The polymerizable liquid crystal compound of claim 1, wherein each of L¹, L², L³ and L⁴ independently represents a alkyl group or hydrogen atom, and at least one of L¹, L², L³ and L⁴ represents a C₁₋₄ alkyl group.

4. The polymerizable liquid crystal compound of claim 1, wherein each of L¹, L², L³ and L⁴ independently represents a methyl group or hydrogen atom, and at least one of L¹, L², L³ and L⁴ represents a methyl group.

5. The polymerizable liquid crystal compound of claim 1, wherein each of L¹, L², L³ and L⁴ independently represents a methyl group or hydrogen atom, one of L¹, L², L³ and L⁴ being assigned to a methyl group and three of L¹, L², L³ and L⁴ being assigned to hydrogen atoms.

6. The polymerizable liquid crystal compound of claim 1, wherein Z¹ represents a single bond.

7. The polymerizable liquid crystal compound of claim 1, wherein A¹ represents a C₃₋₆ methylene group.

8. The polymerizable liquid crystal compound of claim 1, wherein A¹ represents a C₄ methylene group.

9. The polymerizable liquid crystal compound of claim 1, wherein Z² represents —CO—.

10. The polymerizable liquid crystal compound of claim 1, wherein R¹ represents a hydrogen atom.

11. The polymerizable liquid crystal compound of claim 1, wherein R² represents a C₁₋₄ straight-chain alkyl group, phenyl group, acryloylamino group or methacryloylamino group.

12. The polymerizable liquid crystal compound of claim 1, wherein R² represents a phenyl group, acryloylamino group or methacryloylamino group.

13. A liquid crystal composition comprising at least one polymerizable liquid crystal compound represented by the formula (1) below, and
at least one polymerizable liquid crystal compound represented by the formula (3) below: 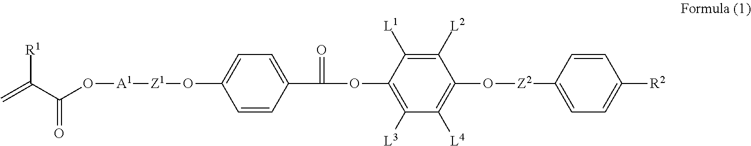
wherein A¹ represents a C₂₋₁₈ methylene group, one CH₂ or two or more non-adjacent (CH₂)s in the methylene group may be substituted by —O—;
Z¹ represents —CO—, —O—CO— or a single bond;
Z² represents —CO— or —CO—CH═CH—;
R¹ represents a hydrogen atom or methyl group;
R² represents hydrogen, C₁₋₄ straight-chain alkyl group, C₁ or C₂ straight-chain alkoxy group, phenyl group, aryloxy group, vinyl group, acryloylamino group, methacryloylamino group, N-aryloxycarbamoyl group, N-alkyloxycarbamoyl group having a C₁₋₄ alkyl group, N-(2-methacryloyloxyethyl)carbamoyloxy group or N-(2-acryloyloxyethyl)carbamoyloxy group; and
each of L¹, L², L³ and L⁴ independently represents a C₁₋₄ alkyl group, C₁₋₄ alkoxy group, C₂₋₅ alkoxycarbonyl group, C₂₋₄ acyl group, halogen atom or hydrogen atom, at least one of L¹, L², L³ and L⁴ represents a substituent other than hydrogen atom. 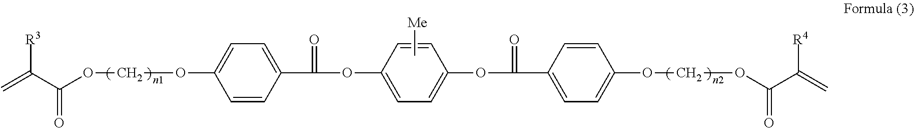
wherein each of n1 and n2 independently represents an integer of 3 to 6; and
each of R³ and R⁴ independently represents a hydrogen atom or methyl group.

14. The liquid crystal composition of claim 13, wherein in the formula (1), R² represents hydrogen atom, C₁₋₄ straight-chain alkyl group, C₁ or C₂ straight-chain alkoxy group, or phenyl group.

15. The liquid crystal composition of claim 13, wherein the polymerizable liquid crystal compound represented by the formula (1) is a polymerizable liquid crystal compound represented by the formula (2) below: 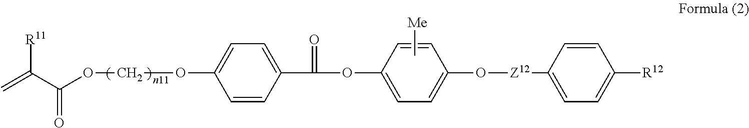
wherein n¹¹ represents an integer of 3 to 6;
R¹¹ represents a hydrogen atom or methyl group;
Z¹² represents —CO— or —CO—CH═CH—;
R¹² represents a hydrogen atom, C₁₋₄ straight-chain alkyl group, C₁ or C₂ straight-chain alkoxy group, phenyl group, aryloxy group, vinyl group, acryloylamino group, methacryloylamino group, N-aryloxycarbamoyl group, N-alkyloxycarbamoyl group having a C₁₋₄ alkyl group, N-(2-methacryloyloxyethyl)carbamoyloxy group, or N-(2-acryloyloxyethyl)carbamoyloxy group.

16. The liquid crystal composition of claim 15, wherein n11 is 4.

17. The liquid crystal composition of claim 15, wherein R¹¹ represents a hydrogen atom.

18. The liquid crystal composition of claim 15, wherein Z¹² represents —CO—.

19. The liquid crystal composition of claim 15, wherein R¹² represents a C₁₋₄ straight-chain alkyl group or phenyl group.

20. The liquid crystal composition of claim 15, wherein R² represents a phenyl group.

21. The liquid crystal composition of claim 13, which comprises the polymerizable liquid crystal compound represented by the formula (1) in a content of 3 to 50% by mass, relative to the polymerizable liquid crystal compound represented by the formula (3).

22. The liquid crystal composition of claim 13, which comprises the polymerizable liquid crystal compound represented by the formula (1) in a content of 5 to 40% by mass, relative to the polymerizable liquid crystal compound represented by the formula (3).

23. The liquid crystal composition of claim 13, containing at least one species of polymerization initiator.

24. The liquid crystal composition of claim 13, containing at least one species of chiral compound.

25. A film comprising an optically anisotropic layer, wherein the optically anisotropic layer is obtainable by fixing alignment of polymerizable liquid crystal compounds described in claim 1.

26. The film of claim 25, wherein the optically anisotropic layer is obtainable by fixing cholesteric alignment of the liquid crystal compounds.

27. The film of claim 26, having selective reflectivity.

28. The film of claim 26, having selective reflectivity in an infrared wavelength region.

29. The film of claim 25, wherein the optically anisotropic layer is obtainable by fixing homogeneous alignment of the liquid crystal compounds.

30. The film of claim 25, wherein the optically anisotropic layer is obtainable by fixing homeotropic alignment of the liquid crystal compounds.

31. A polarizing plate comprising a film described in claim 29 and a polarizing film.

32. A liquid crystal display device comprising a polarizing plate described in claim 31.
